(19) United States
(12) Patent Application Publication
(10) Pub. No.: US 2005/0084963 A1
(43) Pub. Date: Apr. 21, 2005

(54) Purification of lineage-specific cells and uses therefor

(76) Inventor: Tailoi Chan-Ling, Russell Lea (AU)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/479,500

(22) PCT Filed: May 31, 2002
(86) PCT No.: PCT/AU02/00704

Related U.S. Application Data
(60) Provisional application No. 60/295,455, filed on Jun. 1, 2001.

(51) Int. Cl. C12N 5/08

(52) U.S. Cl. 435/368

(57) ABSTRACT

A method for developing a population of substantially lineage-specific cells and their use inter alia in tissue replacement therapy, tissue augmentation therapy, diagnostic applications, for the identification of growth factors and other autocrine factors. Specifically, substantially homogeneous populations of mammalian cells of the astrocyte lineage are provided and selected on the basis of differential marker expression.

### FIELD OF THE INVENTION

The present invention relates generally to a method for developing a population of substantially lineage-specific cells and their use inter alia in tissue replacement therapy and tissue augmentation therapy and in diagnostic applications and for the identification of growth factors and other autocrine factors such as expansion, proliferation and differentiation factors. More particularly, the present invention provides mammalian cells of the astrocytic lineage cells obtainable from mammalian brains such as from embryo brain tissue or parts thereof such as the retina and selected on the basis of differential marker expression. The ability to selectively enrich or obtain or otherwise generate a pure homogeneous population and preferably a pure population of astrocyte precursor cells and immature perinatal astrocytes permits tissue replacement and augmentation therapy of the brain resulting from a degenerative and in particular a neurodegenerative or other disease conditions or trauma. The cells may be derived from the subject to be treated (i.e. autologous transplantation/augmentation therapy) or may be derived from suitably histocompatibility matched individuals (heterologous or non-autologous transplantation/augmentation therapy). The identification of markers specific for certain developmental stages of astrocytic lineage along its maturation pathway permits the development of assays to distinguish between the developmental stages and this is useful in the development of diagnostic and therapeutic tools. Factors identified and obtainable from cultured astrocyte precursor cells or immature perinatal astrocytes are useful in facilitating tissue replacement and augmentation therapy and in inducing tissue repair and regeneration. These factors may also be administered directly into the brain or other suitable location to facilitate development of lineage-specific cells.

### BACKGROUND OF THE INVENTION

Reference to any prior art in this specification is not, and should not be taken as, an acknowledgment or any form of suggestion that this prior art forms part of the common general knowledge in any country.

Multipotent stem cells are undifferentiated cells which are capable of differentiation and proliferation into multiple cell lineages and types and have the ability of self-renewal.

During development of the central nervous system (CNS), multipotent stem cells which have the capacity of generating many types of neurons and glia, give rise to precursor cells that are progressively more restricted in differentiation potential. Although substantial progress has been made in understanding the development of oligodendrocytes and Schwann cells, much less is known about astrocyte development. Furthermore, whereas several types of multipotent stem cells and lineage-restricted precursor cells have been characterized and applied clinically in recent years, the lack of knowledge of the sequence of events that underlies astrocyte development has limited the success of such applications.

It has been shown that a glial-restricted precursor (GRP) cell isolated from the spinal cord of mice on embryonic day (E) 13.5 is capable of giving rise to oligodendrocytes and both type-1 and type-2 astrocytes in culture. However, the intermediate stages of differentiation between the GRP cell and mature, differentiated astrocytes present in the adult CNS are not well characterized. There is a need, therefore, to increase the understanding of the developmental biology of cells of the astrocytic lineage.

Early studies provided evidence for the existence of astrocyte precursor cells (APCs) that give rise only to astrocytes (Raff et al., Devel. Biol. 106: 53-60, 1984; Fok-Seang, J. and Miller, R. H., J. Neurosci. 12: 2751-2764, 1992; Davis, A. A. and Temple, S., Nature 372: 263-266, 1994; Levison, S. W. and Goldman, J. E., J. Neurosci. Res. 48: 83-94, 1997; Mi. J. and Barres. B. A., J. Neurosci. 19: 1049-1061, 1999). Such cells present in cultures of neonatal rat spinal cord were characterized as highly migratory as well as positive for the A2B5 antigen and vimentin and negative for glial fibrillary acidic protein (GFAP) and galactocerebroside (Fok-Seang and Miller, 1992, supra). Such cells are also present in neonatal rat optic nerve and were characterized as positive for Pax2, A2B5, C5, Ran-2 and Vimentin and negative for GFAP, S100β, and weakly positive for nestin (Ni and Barres, 1999, supra).

Little is known of the characteristics of APCs in vivo. Until the advent of the present invention, the existence of an APC that gives rise only to astrocytes in the developing human CNS and adult CNS has not previously been demonstrated. Furthermore, immunohistochemical and in situ hybridization analyses have shown that, in the mouse cerebellum, Pax2 (Mi and Barres, 1999, supra) is not expressed by cells of the astrocytic or oligodendrocytic lineages, but is rather localized to γ-aminobutyric acid-containing interneurons and deep cerebellar nuclei (Maricich, S. M. and Herrup, K., J Neurobiol. 41: 281-294, 1999). There is an apparent discrepancy, therefore, in Pax2 gene expression data following in vitro and in vivo studies of CNS development. Furthermore, until the advent of the present invention, there was a lack of in vivo studies of APC differentiation in human fetal tissue.

The Pax2 gene is a member of the Pax gene family which encodes transcription factors, all of which are DNA-binding proteins that contain a paired-box domain. Each member of the Pax family is expressed in a spatially and temporally restricted manner, suggesting that these proteins contribute to the control of tissue morphogenesis and pattern formation. Pax2 is implicated in organogenesis of the kidney, eye, ear, and the CNS. Heterozygous mutations in the Pax2 gene result in failure of the optic groove to form in the mouse optic nerve (Otteson et al., Devel. Biol. 193: 209-224, 1998) and are associated in humans and mice with optic nerve coloboma (Sanyanusin et al., Nature Genetics 9: 358-364, 1995; Favor et al., Proc. Natl. Acad. Sci. USA 93: 13870-13875, 1996), a condition characterized by enlargement and blurring of the margin of the optic disk. Homozygous mutations in the Pax2 gene result in retinal coloboma as a consequence of failure of the retinal fissure to close (Torres et al., Development 122: 3381-3391, 1996).

Pax2 expression during ocular development has been studied in mice, rats, and humans. Transcripts of the Pax2 gene are first apparent in the developing mouse eye on embryonic day (E) 9 and are initially restricted to the ventral optic cup and stalk (Nornes et al., Development 109: 797-809, 1990; Otteson et al., 1998, supra). By E16.5, these transcripts have disappeared from the ventral retina and are present in a ring of cells around the optic nerve head (ONH) and in the parenchyma of the optic nerve. At E18, Pax2 mRNA is apparent on the vitreal surface of the posterior retina, consistent with the timing and topography of astrocyte migration into the mouse retina. Pax2 mRNA was not detected in the retina, optic disk, or optic nerve of adult mice (Otteson et al., 1998, supra). In the rat optic nerve, Pax2 expression is already widespread at E17 (Mi and Barres, 1999, supra). Although the pattern of Pax2 expression during development of the rat optic nerve is consistent with the observations in the mouse, Pax2 expression persists at a low level in the adult rat nerve. Limited observations in humans have shown that, between 6 and 8 weeks of gestation (WG), Pax2 is expressed in the region of the optic disk and nerve (Terzic et al., Int. J. Dev. Biol. 42: 701-707, 1998). Despite the documentation of Pax2 expression during early embryonic development of the optic nerve and eyecup until the advent of the present invention, the relation between Pax2 expression and differentiation of the astrocytic lineage during the later stages of retinal development has been unknown.

Various studies have examined the development of GFAP⁺ astrocytes in vivo. In the human retina, astrocytes with two distinct morphologies and locations have been described: those with parallel processes closely associated with nerve fiber bundles (NFBs), and star-shaped astrocytes present in the ganglion cell layer (GCL) that often ensheath blood vessels (Wolter, J., Am. J: Ophthal 40: 88-99, 1955; Ogden, T. E., Invest. Ophthalmol. Vis. Cii. 17: 499-510, 1978; Ramirez et al., Vis. Res. 34: 1935-1946, 1994; Trevino et al., Vis. Res. 37: 1707-1711, 1997; Provis et al., Exp. Eye Res. 65: 555-568, 1997; Hughes et al., Invest. Ophthalmol. Vis. Sci. 41: 1217-1228, 2001). Astrocytes first appear in the monkey retina around the optic disk and spread peripherally, reaching the edge of the retina before birth (Gariano et al, Invest. Ophthalmol. Vis. Sci. 37: 2367-2375, 1996); vimentin and GFAP immunohistochemical analysis of retinal sections revealed that immature spindle-shaped astrocytes precede the developing vasculature. Astrocytes also precede the formation of blood vessels by a small margin in the human (Chan-Ling et al., Proc. Aust. Neurosci. Soc. 7: 48, 1996; Provis et al., 1997, supra; Hughes et al., 2001, supra), cat (Ling, T. and Stone, J., Dev. Brain Res. 44: 73-85, 1988; Chan-Ling, T. and Stone, J., J. Comp. Neurol. 303: 387-399, 1991), and rat (Ling et al., J. Comp. Neurol. 286: 345-352, 1989) retina and are thought to secrete vascular endothelial growth factor (VEGF), which mediates hypoxia-induced angiogenesis (Chan-Ling et al., Invest. Ophthalmol. Vis. Sci. 36: 1201-1214, 1995; Stone et al., J. Neurosci. 15: 4738-4747, 1995; Hughes et al, 2000, supra).

In work leading to the present invention, the present inventor investigated the relationship between Pax2 expression and cells of the astrocytic lineage in the human retina and the optic nerve head (ONH), and characterized the time course of appearance and topography of spread of APCs and perinatal astrocytes in the human retina.

The subject inventor identified positive and negative markers which were specific for particular developmental stages during maturation of astrocytic lineage. In accordance with the present invention, these markers in combination with other in vitro markers, are used to selectively enrich or generate populations of APCs or immature perinatal astrocytes (IPAs) or other astrocyte cells such as mature perinatal astrocytes. Furthermore, in accordance with the present invention, APCs are identified in the adult human brain. The ability to generate such populations permits their developmental expansion for use in tissue replacement and augmentation therapy and to identity factors involved in their proliferation and differentiation. The identification of immature APCs in the adult human brain is particularly significant in terms of a source of cells for autologous therapy. Furthermore, the present invention encompasses antagonists and agonists of these factors as well as naturally occurring molecules which inhibit proliferation, differentiation and/or growth of these cells.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The present invention identifies astrocyte cell markers which are capable of distinguishing between developmental stages. From multipotent stem cells, lineage-specific APCs are formed expressing Pax2 and vimentin but not either glial fibrillary acid protein (GFAP) or S-100. The next developmental stage is the formation of IPAs which express all four of the above markers. Mature perinatal astrocytes (MPAs) lose the ability to express vimentin and then adult astrocytes further lose Pax2 expression as a function of physiologic aging. The ability to selectively enrich cultures of cells for APCs or IPAs permits their use in tissue replacement and augmentation therapy. Importantly, APCs have been identified in accordance with the present invention in adult brain as well as the retina and, hence, this aspect represents a source of APCs for autologous therapy as well as for heterologous therapy. Furthermore, homogeneous populations of APCs or IPAs can be used to isolate particular growth or autocrine factors for use in conjunctive therapy to tissue replacement and augmentation therapy or to induce repair or regeneration of endogenous tissue. The markers further permit mixed populations of astrocytes in various stages of development to be identified and this has diagnostic and therapeutic applications.

Accordingly, one aspect of the present invention contemplates a method for generating a substantially homogeneous population of lineage-specific cells from tissue of the central nervous system (CNS) of mammalian animals, said method comprising subjecting said CNS tissue to tissue disruptive means comprising the lineage-specific cells to be isolated, subjecting the mixed population to cell separation discrimination means to generate a substantially homogeneous population of lineage-specific cells.

Preferably, the lineage-specific cells are APCs or IPAs from tissue of the CNS such as brain including retina tissue.

The present invention contemplates, therefore, a method of generating a substantially homogeneous population of APCs from tissue of the CNS such as from brain or parts thereof including the retina or parts thereof, said method comprising subjecting said CNS tissue to tissue disruptive means to produce a population comprising APCs amongst other cells and subjecting said population of cells to a cell sorting methodology including such as subjecting cells to positive selection using surface markers GD3, A2B5, C3B2, FGFR3 and/or PDGFRα or a combination thereof, then subjecting the positively selective cells to negative selection using GlC, 01, 04, anti-Mog and/or NG2 or a combination thereof. The identity of the purified population of cells is confirmed using Pax2, vimentin, GFAP and S-100 immunohistochemically.

Having obtained the APCs, a substantially homogenous population of IPAs or a mixed population of IPAs and APCs are included along the mature pathway is induced using one or a combination of inter alia CNTF, LIF, BMP (e.g. BMP4), TGFβ, cAMP and EGF.

In a most preferred embodiment, the cells are purified as follows. A population of cells is selected and single cell suspensions prepared. Using negative selection such as N-CAM (also known as PSA-N-CAM neural cells are removed from their cell population. Glial cells are positively selected using markers such as A2B5, GD3, 3CB2, FGFR3, PDGFRα or a combination thereof. The cells are then cultured in a serum free medium such as DMEM/F-12 supplemented with growth factors such as bFGF and chick embryo extract. In the resulting population, oligodendrocytes are removed using markers such as GlC, 01, 04, Gal-C, anti-MOG and NG2. The resulting population is induced to differentiate along the maturation pathway using growth factors such as CNTF, LIF, BMP such as BMP4, cAMP, TGFβ and EGF. The cells can then be characterized immunohistochemically based on the markers presented in Table 1.

The present invention provides, therefore, a substantially homogeneous population of mammalian lineage-specific cells from the CNS. The preferred mammalian lineage-specific cells are APCs or IPAs.

Another aspect of the present invention contemplates a method of cell replacement therapy in a mammalian animal, said method comprising generating a substantially homogeneous population of lineage-specific cells and introducing same into an organ or tissue requiring cells to be replaced or to another location from where the cells can migrate to an organ or tissue requiring cells wherein the introduced cells are subject to expansion or proliferation in vitro and/or in vivo by one or more growth factors. Generally, the lineage-specific cells are APCs or IPAs. The APCs or IPAs or tissues derived therefrom may be from the subject being treated (i.e. autologous therapy) or from a suitably histocompatibility matched subject (i.e. heterologous therapy). Autologous therapy is preferred.

Yet another aspect of the present invention provides a composition of astrocyte precursor cells such as APCs or IPAs in substantially homogeneous form, said composition optionally further comprising one or more pharmaceutical acceptable carriers and/or diluents.

Still another aspect of the present invention contemplates a growth or autocrine factor obtainable from conditioned medium of an in vitro cell culture of astrocyte precursors such as APCs or IPAs. The growth or autocrine factor may be used in vitro to expand a population of lineage-specific cells or may be administered directly to the brain to facilitate or promote development of replacement cells.

In a related embodiment, the present invention proposes the use of microarray technology and differential expression arrays to determine cell surface markers including differentially expressed cell surface markers at different stages of astrocyte cell development. Such studies assist in the identification of growth factor receptors for use in selecting growth and autocrine factors to promote proliferation and/or differentiation of particular astrocyte cells.

A further aspect of the present invention contemplates the use of the purified astrocytes and in particular APCs and IPAs as gene therapy carriers.

### BRIEF DESCRIPTION OF THE FIGURES

FIG. 1 is a photographic representation showing: (A) Cryostat section of a human retina at 24 to 26 weeks gestation labeled with both anti-Pax2 (red) and anti-GFAP (green). Pax2⁺, GFAP⁻ APCs (arrow) and Pax2⁺, GFAP⁺ perinatal astrocytes (arrowhead) were detected only within the NFL and GCL. Pax2 expression was apparent only in the cell somas. Autofluorescent granules were observed in the RPE. (B and C) Retinal whole-mounts triple-labeled with anti-Pax2 (red), anti-vimentin (green), and anti-GFAP (blue) at 12 weeks gestation. (B) At the posterior pole, APCs were Pax2⁺, GFAP⁻, and vimentin⁺ (arrow), whereas perinatal astrocytes were Pax2⁺, GFAP⁺, and vimentin⁺ (arrowheads). (C) At the leading edge of Pax2 expression, APCs were Pax2⁺, GFAP⁻, and vimentin⁺ (arrows). (D and E) Retinal whole-mount at 32 weeks gestation double-labeled for Pax2 (red) and GFAP (green). (D) Toward the retinal periphery, Pax2⁺, GFAP⁺ perinatal astrocytes with bipolar processes were located in superficial layers. (E) Vascular layer of the same region of the retina as that shown in (D), revealing Pax2⁺, GFAP⁺ perinatal astrocytes closely associated with blood vessels. (F) Posterior region of a retinal whole-mount at 32 weeks gestation with Pax2 (red) and vimentin (red) labeling. Vimentin filaments (arrows) were restricted to only a few Pax2⁺ cells. (G) Retinal whole-mount at 17 weeks gestation labeled with anti-GFAP (brown). Perinatal astocytes were closely associated with NFBs. (H) Retinal whole-mount at 17 weeks gestatation double-labeled with anti-CD34 (pink) and anti-GFAP (brown). At the leading edge of vessel formation, perinatal astrocytes preceded the formation of patent blood vessels by a small margin. Color versions of this photograph are available from the patentee.

FIG. 2 is a photographic representation showing: (A through F) Cryostat section of a retina at 24 to 26 weeks gestation labeled with both anti-Pax2 (red) and anti-GFAP (green). Posterior (A and B), equatorial (C and D), and peripheral (E and F) regions are shown. APCs (arrows) and perinatal astrocytes (arrowheads) are indicated. (A and B) Pax2⁺, GFAP⁻ APCs were observed in the superficial layer of the NFL. (E) Only Pax2⁺, GFAP⁻ APCs were apparent peripherally. (F) At the retinal edge, neither Pax2⁺, GFAP⁻ APCs nor Pax2⁺, GFAP⁺ perinatal astrocytes were detected. (G) Photographic montage of the ONH region of a retina at 8 weeks gestation labeled with both anti-Pax2 (red) and anti-GFAP (green). (H) A tracing of the montage in (G) showing the location of individual Pax2⁺, GFAP⁻ APCs and Pax2⁺, GFAP⁺ perinatal astrocytes. Two clusters of APCs and perinatal astrocytes are present at the ventricular zone surrounding the ONH. (and J) Adjacent sections of a retina at 24 to 26 weeks gestation double-labeled with anti-Pax2 (red) and anti-GFAP (green) (I), or stained with toluidine blue (J), showing the ventricular zone at high magnification. (I) APCs (arrow) and perinatal astrocytes (arrowhead) were observed in the ventricular region. Autofluorescent granules were apparent in the RPE. Color versions of this photograph are available from the patentee.

FIG. 3 is a photographic representation showing: (A through C) Retinal whole-mounts positioned with the RPE uppermost and showing triple labeling of the ONH region at 14 to 16 weeks gestation with anti-Pax2 (red), anti-vimentin (green), and anti-GFAP (blue). (A) The ONH is located at the top left of the image. At the ventricular zone, the ONH is surrounded by Pax2⁺, GFAP⁺, vimentin⁺ perinatal astrocytes (arrowhead) and an outer layer of Pax2⁺, GFAP⁻, vimentin⁺ APCs (arrow). (13) Higher magnification of the region in (A), showing the layers of APCs and perinatal astrocytes. (C) Higher magnification of the region in (A), showing the outer layer of Pax2⁺, GFAP⁻, vimentin⁺ APCs. (D) The ventricular zone of a retinal whole-mount at 32 weeks gestation double-labeled with anti-Pax2 (red) and anti-GFAP (green). The ONH is located beyond the top left corner of the image. It is still surrounded by Pax2⁺, GFAP⁺ perinatal astrocytes and an outer layer of Pax2⁺, GFAP⁻ APCs. (E through G) Schematic representations of the distributions of Pax2⁺, GFAP⁻, vimentin⁺ APCs and Pax2⁺, GFAP⁺, vimentin⁺ perinatal astrocytes around the ONH at 14 to 16 weeks gestation (E), 24 to 26 weeks gestation (F), and 32 weeks gestation (G). Color versions of this photograph are available from the patentee.

FIG. 4 is a graphical representation of topographic maps of the outer limits of APCs and perinatal astrocytes in the human retina at 12, 16, 18, 21, 22 to 23, 26, 28, and 32 weeks gestation as well as the distributions of perinatal astrocytes and adult astrocytes in the aged adult human retina. In the fetal retinas, red dots indicate the area of APCs and purple dots show the area of perinatal astrocytes. With the exception of a rim at the leading edge of astrocyte migration, where only APCs are found, both APCs and perinatal astrocytes are present interspersed over the central region of the retina during fetal development. The macular region does not contain either of these cell types. The distributions of Pax2⁺, GFAP⁺, vimentin mature perinatal astrocytes (purple dots) and Pax2⁻, GFAP⁺, vimentin⁻ adult astrocytes (blue dots) in the aged adult human retina are shown in the bottom two maps. Scale bars, 1 mm (12 weeks gestation) or 10 mm (all other ages). Color versions of this photograph are available from the patentee.

FIG. 5 is a photographic representation showing (A and B) Retinal whole-mount at 16 weeks gestation triple-labeled with anti-Pax2 (red), anti-vimentin (green), and anti-GFAP (blue). (A) At the leading edge of Pax2 labeling, APCs were Pax2⁺, vimentin⁺, and GFAP⁻. (B) Higher magnification of the same region of the retina shown in (A). (C and D) The foveal and raphe regions of a retinal whole-mount at 18 weeks gestation double-labeled with anti-Pax2 (red) and anti-GFAP (green). (C) Arrowheads indicate the border of the presumptive fovea. Pax2⁺, GFAP⁻ APCs and Pax2⁺, GFAP⁺ perinatal astrocytes were not detected in the presumptive foveal zone. Small arrow points towards representative APCs and large arrow points towards a perinatal immature astrocyte in the perifoveal region. (D) Higher magnification of the boxed region in (C), showing the border zone. (E and F) The foveal and raphe regions of retinal whole-mounts at 18 weeks gestation labeled with anti-GFAP (brown). Perinatal astrocytes follow the path of NFBs in the raphe region. (G) Adult retinal whole-mount triple-labeled with anti-Pax2 (red), anti-vimentin (red) and anti-GFAP (green) and showing the presence of Pax2⁺, GFAP⁺, vimentin⁻ mature perinatal astrocytes (arrow) at the posterior pole. (H) Whole-mount of a second adult retina subjected to triple-label immunohistochemistry as in (G). GFAP⁺ astrocytes did not express Pax2. Color versions of this photograph are available from the patentee.

### ABBREVIATIONS

| ABBREVIATION | DESCRIPTION |
|---|---|
| APCs | astrocyte precursor cells |
| IPAs | immature perinatal astrocytes |
| MPAs | mature perinatal astrocytes |
| GFAP | glial fibrillary acid protein; expressed in all astrocyte |
| | cells except APCs |
| CNS | central nervous system |
| FACS | fluorescence activated cell sorter |
| Pax2 | marker expressed in APCs, IPAs and MPAs and a |
| | proportion of adult astrocytes |
| vimentin | marker expressed in APCs and IPAs |
| DTPA | diethylenetriaminepentacetic acid |
| EDTA | ethylenediaminetetracetic acid |

### DETAILED DESCRIPTION OF THE PREFERRED EMBODIMENTS

The present invention is predicated in part on the use of markers to selectively enrich lineage-specific cells from mammalian brains. The mammalian brains may be from a prenatal stage (e.g. an embryo) or from a postnatal animal including an adult. It is particularly significant that APCs have been identified, in accordance with the present invention, from adult brains. Reference herein to a brain includes parts thereof such as the retina or parts thereof. Even more particularly, the markers distinguish the four developmental stages of astrocyte maturation which are astrocyte precursor cells (APCs), immature perinatal astrocytes (IPAs), mature perinatal astrocytes (MPAs) and adult amd aged astrocytes.

Although in many circumstances, it is desirable to use immature cells, i.e. APCs and IPAs, the present invention extends to the use of all astrocyte types including MPAs and adult astrocytes.

In accordance with the present invention, the four stages of astrocyte development may be characterized by differential marker expression as described below in Table 1:—

TABLE 1

| | Astrocyte cell types | | | | |
|---|---|---|---|---|---|
| Marker | APCs | IPAs | MPAs | Adult astroycte | Aged astrocyte |
| Pax2 | + | + | + | −/+ | −/+ |
| GFAP | − | + | + | + | + |
| vimentin | + | + | − | − | − |
| S-11 | − | + | + | + | + |

Accordingly, one aspect of the present invention contemplates a method for generating a substantially homogeneous population of lineage-specific cells from tissue of the central nervous system (CNS) of mammalian animals, said method comprising subjecting said CNS tissue to tissue disruptive means to provide a mixed population of cells comprising the lineage-specific cells to be isolated, subjecting the mixed population to cell separation discrimination means to generate a substantially homogeneous population of lineage-specific cells.

A number of cell isolation, cell separation, and cell purging strategies are known for purifying or removing cells from a suspension comprising a diverse population of cells. Cell separation methods that are used to isolate cells or purge cell suspensions or cell populations typically fall into one of three broad categories. Physical separation methods typically exploit differences in a physical property between cell types, such as cell size or density (e.g. centrifugation); chemical-based methods typically employ an agent that selectively kills or purges one or more undesirable cell types; and affinity-based methods typically exploit antibodies or molecules with a selective binding capacity that bind selectively to marker molecules on or in a cell membrane surface or on or in a cell of desired or undesired cell types, which antibodies may subsequently enable the cells to be isolated or removed from the suspension. It is not intended that the method of purification of cells of astrocyte precursor cells be limited to any one method. However, cell marker separation means is the most convenient to date and, in particular, sorting of cells by immunological recognition of cell markers.

Once the starting source of CNS tissue is obtained, astrocyte cells can be removed, and thus selectively separated and purified, by various methods which preferably utilize antibodies and cell markers. In these methods, antibodies or molecules that selectively bind to specific marker molecules present on or in, for example, the astrocyte precursor cells of interest, but do not bind to other cells within the source material. The bound molecule then acts as a flag to signal the identification of the appropriate cell type.

Cell types of non-astrocytic lineage can be removed using negative selection (e.g. by immunopaning) as follows: N-CAM (PSA-N-CAM), 01, 04, GlC, Gal-C, NG2 and anti-mog for removal of oligodendrocytes and their precursors; CD31 and CD34 for removal of vascular endothelial cells and other markers for removal of fibroblasts.

These techniques can include, for example, flow cytometry using a fluorescence activated cell sorter (FACS) and specific fluorochromes, biotin-avidin or biotin-streptavidin separations using biotin conjugated to cell marker-specific antibodies and avidin or streptavidin bound to a solid support such as affinity column matrix or plastic surfaces or magnetic separations using antibody-coated magnetic beads. Reference to “astrocyte cells” includes reference to all forms of cells including APCs, IPAs, MPAs and adult astrocytes and aged astrocytes. These are all encompassed by reference to astrocytic lineage. Depending on the condition to be treated, the selection of which type of astrocyte cell can be made. Astrocyte cell hybrid may also be employed using different astrocyte cells or astrocyte cells with different neural cells.

In an alternative method, which is particularly efficient for the practice of the present invention, a negative selection protocol is adopted. In a negative selection, the markers are on cell types of interest of astrocytic lineage. Consequently, non-astrocyte cells are removed or astrocyte cells of not the desired level of maturity or immaturity are removed.

Therefore, separation via cell marker discrimination utlilizes antibodies or other molecules that selectively bind specific markers and can be achieved by negative or positive selection procedures. In negative separation, antibodies are used which are specific for markers present on or in undesired cells, as for example, in the case of an astrocyte precursor population, where it would be desirable to deplete the number of non-precursor cells. In this case, antibodies could be directed to the extracellular domain of proteins not present on or in the precursor cells. Cell markers suitable for such a method of cell discrimination include but are not limited to positive markers such as GD3 and A2B5 and negative markers such as GlC, 04, NG2. Pax2, GFAP and vimentin represent useful histochemical markers. Alternatively, it may be desirable to directly select the desired cells from a population of cells. In this case, antibodies or other molecules that selectively bind an extracellular domain of a cell protein can be used. Cells bound by such an antibody to a cell marker can then be sorted and the remaining cells removed and the desired mixture retained. Cell markers suitable for such a method of cell discrimination include but are not limited to GD3 and A2B5.

The cell markers used for cell discrimination means may be labeled with a fluorescent compound. When the fluorescently labeled antibody or molecule with selective binding capacity is exposed to light of the proper wavelength, its presence can then be detected due to fluorescence. Among the most commonly used fluorescent labeling compounds are fluorescein isothiocyanate, rhodamine, phycoeryirin, phycocyanin, allophycocyanin, o-phthaldehyde and fluorescamine. The antibody or molecule with selective binding capacity can also be detectably labeled using fluorescence emitting metals such as ¹⁵²Eu, or others of the lanthanide series. These metals can be attached to the antibody or molecule with selective binding capacity using such metal chelating groups as diethylenetriaminepentacetic acid (DTPA) or ethylenediaminetetraacetic acid (EDTA). The antibody also can be detectably labeled by coupling it to a chemiluminescent compound. The presence of the chemiluminescent-tagged antibody or molecule with selective binding capacity is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester. Likewise, a bioluminescent compound can be used to label the antibody or molecule with selective binding capacity of the present invention. Bioluminescence is a type of chemiluminescence found in biological systems, in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin. All such methods of labeling an antibody or a molecule with selective binding capacity are contemplated by the present invention.

The cell marker discrimination means includes contacting the cells to at least one cell marker at least resident on the lineage-specific cells to be isolated and using same to isolate such cell types and then using another molecule interactive with at least one other marker either on the cell of interest or on cells to be discarded to enrich for the desired lineage-specific cells.

The above method may be varied to use the first contact with an interactive molecule to discard cells not intended to be isolated.

Tissue of the CNS includes the brain or parts thereof including the retina. The retina is considered part of the brain and is connected to the brain via the optic nerve.

The lineage-specific cells particularly preferred in accordance with the present invention are astrocyte cells and, most preferably, are APCs or IPAs. However, any astrocyte cell may be isolated according to the methods of the present invention.

Accordingly, another aspect of the present invention provides a method for isolating APCs or IPAs from tissue of the CNS such as brain including retina tissue, said method comprising subjecting said CNS tissue or part thereof to tissue disruptive means to provide a mixed population of cells comprising the APCs or IPAs to be isolated, subjecting the cells to interactive molecules to a cell marker selectively present or absent on or in or in said APCs or IPAs to generate a population comprising at least APCs and/or IPAs and then contacting the isolated cells with an interactive molecule to at least one other cell marker specific for either said APCs or IPAs or specific for a cell marker absent from either APCs or IPAs to selectively remove or retain the desired cell type.

Where the desired cell types are APCs or IPAs, APCs are the most preferred. Other forms of astrocytes such as MPAs or adult astrocytes may also be useful and are encompassed by the present invention. For tissue replacement therapy, however, APCs or IPAs may be used but APCs are most preferred.

The selection of particular cell types is based on the differential expression of cell markers and, in particular, those selected from Pax2, GFAP, S-100 and vimentin in or on APCs, IPAs, MPAs and adult astrocytes and aged astrocytes as described in Table 1.

The preferred interactive molecules are immunoglobulins and are referred to herein as immunointeractive molecules. The term “immunoglobulins” encompasses antibodies or antigen-specific binding portions thereof such as Fab fragments. The term “immunoglobulins” or “antibodies” further encompasses synthetic or recombinant or hybrid forms of these molecules.

Accordingly, in a preferred embodiment, the present invention contemplates a method of generating a substantially homogeneous population of APCs from tissue of the CNS such as from brain or parts thereof including the retina or parts thereof, said method comprising subjecting said CNS tissue to cell disruptive means to produce a population comprising APCs amongst other cells and subjecting said population of cells to specific immunological separation using a GD3⁺-positive and/or A2B5⁺-positive selection technique and then a negative selection comprising GlC, 01, 04, Gal-C, NG2 and/or anti-Mog cells to generate a population of APCs. In a preferred embodiment, following the positive selection, the cells are cultured in a suitable culture medium such as serum free medium, for example, DMEM/F-12 together with growth factors such as bFGF or chick embryo extracts. The oligodendrocytes are then removed using the negative selection. The purity of these cells is determined and/or confirmed using a combination of a Pax2, vimentin, GFAP and S-100 immunohistochemically. APCs are Pax2⁺, GFAP⁻, S-100⁻ and vimentin⁺. One skilled in the art will immediately recognize that other markers may be employed and all such differentiating markers are encompassed by the present invention.

In another preferred embodiment, the present invention is directed to a method of generating a substantially homogeneous population of IPAs from the APCs described above by exposing said APCs to CNTF, LIF, BMP including BMP4, TGFβ, cAMP and/or EGF to induce GFAP expression and differentiation along the maturation pathway.

Given the range of differential marker expression on astrocyte cells, one skilled in the art will readily recognize the ability to select specifically any cell type such as APCs, IPAs, MPAs or adult astrocytes. Furthermore, there may be a number of alternative combinations of cell markers which would be equally efficacious in isolating the one desired cell type.

Reference herein to a “population” of cells means two or more cells. A “homogeneous population” means a population comprising substantially only one cell type. A “cell type” may be cells of the same lineage or sub-type having substantially the same physiological status. Preferred homogeneous populations comprise substantially only APCs or IPAs or MPAs or adult astrocytes or aged astrocytes. In terms of tissue replacement or augmentation therapy, the cells may be derived from the subject to be treated (autologous therapy) or from a suitably histocompatibility matched undivided (heterologous or non-autologous therapy).

Reference herein to a “substantially homogeneous population” refers to a cell population in which a substantial number of the total population of the cells are of the same type and/or are in the same state of differentiation. Preferably, a “substantially homogeneous population” of astrocyte cells comprises a population of cells of which at least about 50% are of the same cell type (e.g. APCs, IPAs, MPAs or adult astrocytes), more preferably that at least about 75% are of the same cell type, even more preferably at least about 85% are of the same cell type, still even more preferably at least about 95% of the cells are the same type, and even more preferably at least about 97% (e.g. 98%, 99% or 100%) are of the same cell type.

The term “tissue-disruption means” includes dissociation of individual cells from the connecting extracellular matrix (ECM) of the CNS tissue. Preferably, a single cell suspension is produced.

The preferred cells are generally not fully differentiated and, hence, may be regarded as committed (i.e. single lineage) but nevertheless partially undifferentiated.

“Undifferentiated” means a primordial state of a cell or cells capable of differentiation and proliferation to produce progeny cells that can be physiologically, biochemically, morphologically, anatomically, immunologically, physiologically, or genetically distinct from the primordial state. The preferred undifferentiated cells are APCs or IPAs. These cells are capable of differentiation or maturation into MPAs and then adult astrocytes.

The present invention is directed to CNS from mammalian subjects. Such subjects include primates, humans, livestock animals (e.g. sheep, cows, horses, donkeys, goats, pigs), laboratory test animals (e.g. mice, rats, rabbits, guinea pigs, hamsters) and companion animals (e.g. dogs, cats). Preferred animals are humans and laboratory test animals such as rats and mice.

The CNS may be disrupted in vitro and then subjected to immunological separation of particular cells and/or may be immobilized in a solid phase such as frozen sections and/or a gelatin matrix.

In one particular embodiment, CNS tissue is subjected to cryostat treatment and sections cut and mounted onto gelatin coated shades. The sections may then be subjected to immunological testing with individual or combinations of antibodies.

The antibodies contemplated for use in accordance with the present invention may be prepared in any animal such as rabbit, mouse, rat, guinea pig, horse, sheep, pig, amongst a range of other animals or birds, such as chickens or other poultry birds. The antibodies are conveniently directed to synthetically prepared or recombinantly produced or naturally occurring, purified forms of Pax2, GFAP, S-100 or vimentin. Furthermore, structurally or antigenically related molecules may also be employed which elicit antibodies which cross-react with one of Pax2, GFAP, S-100 and vimentin.

The present invention is predicated in part on the use of positive and negative cell selection of cell surface markers such as subjecting cells to positive selection using surface markers GD3, A2B5, C3B2, FGFR3 and/or PDGFRA or a combination thereof, then subjecting the positively selective cells to negative selection using GlC, O4 and/or NG2 or a combination thereof. The identity of the purified population of cells is confirmed using Pax2, vimentin, GFAP and S-100 immunohistochemically.

The present invention provides, therefore, a substantially homogeneous population of mammalian lineage-specific cells from the CNS, said cells made by the method comprising subjecting said CNS tissue to tissue disruptive means to provide a mixed population of cells comprising the lineage-specific cells to be isolated, subjecting the mixed population to cell separation discrimination means to generate a substantially homogeneous population of lineage-specific cells.

More particularly, the present invention is directed to a substantially homogeneous population of APCs or IPAs from the CNS such as brain including retina tissue prepared by the method comprising subjecting said CNS tissue or part thereof to tissue disruptive means and contacting said immobilized tissue with interactive molecules to a cell marker selectively present or absent on or in said APCs or IPAs to generate a population comprising at least APCs and/or IPAs and then contacting the isolated cells with an interactive molecule to at least one other cell marker specific for either said APCs or IPAs or specific for a cell marker absent from either APCs or IPAs to selectively remove or retain the desired cell type.

Preferably, the homogeneous population of cells comprises substantially only APCs or IPAs, prepared as described above.

The ability to generate a substantially homogeneous population of particular astrocyte cells such as APCs or IPAs permits the development of a range of therapeutic applications such as tissue replacement therapy, augmentation therapy (for example, co-transplantation of human stem cells with human APCs and/or IPAs) and therapy to repair, replicate or delay senescence of astrocytes.

Accordingly, another aspect of the present invention contemplates a method of cell replacement therapy in a mammalian animal, said method comprising generating a substantially homogeneous population of lineage-specific cells and introducing same into an organ or tissue requiring cells to be replaced or to another location from where the cells can migrate to an organ or tissue requiring cells wherein the introduced cells are subject to expansion or proliferation in vitro and/or in vivo by one or more growth factors.

Examples of suitable growth factors include inter alia CNTF, LIF, BMP such as BMP4, TGFβ, cAMP and EGF.

More particularly, the present invention contemplates a method of cell replacement or augmentation therapy in a mammalian animal, said method comprising generating a substantially homogeneous population of astrocyte precursor lineage-specific cells and introducing same into an organ or tissue requiring cells to be replaced or to another location from where the cells can migrate to an organ or tissue requiring cells wherein the introduced cells are subject to expansion or proliferation in vitro and/or in vivo by one or more growth factors.

Preferably, the astrocyte precursor is APCs or IPAs.

This method of the present invention is useful for treating a range of degenerative disorders including Alzheimer's disease, HIV-associated dementia (HIVD), Huntington's disease, chronic neurological disorders, Parkinson's disease, epilepsy, stroke or alcoholism. Furthermore, the present method may be used to treat hypoxia or the effects thereof as well as spinal cord injuries. Other conditions contemplated for treatment by the present invention include acute brain injury (e.g. head injury or cerebral palsy) and a large number of CNS dysfunctions (e.g. depression and schizophrenia). In recent years, neurodegenerative disease has become an important concern due to the expanding elderly population which is at greatest risk for these disorders. These diseases, which include Alzheimer's disease, multiple sclerosis (MS), Huntington's disease, amyotrophic lateral sclerosis and Parkinson's disease, have been linked to the degeneration of cells in particular locations of the CNS, leading to the inability of these cells or the brain region to carry out their intended function. The treatment of all such conditions is encompassed by the present invention. Other conditions contemplated herein include Angleman sydrome, Charcot-Marie-Tooth disease, epilepsy, essential tremor, fragile X syndrome, Friedreich's ataxia, Niemann-Pick disease, Prader-Willi syndrome, Rett syndrome, spinocerebella atrophy and William's syndrome as well as other conditions affecting the brain or CNS such as a stroke, alcoholism or drug or other substance abuse. Furthermore, visual and/or cognitive impairment due to, for example, aging dementia may also be treated. Still further, the method of the present invention is useful for augmentation therapy to regenerate aging tissue including co-transplantation of human APCs and/or IPAs with human stem cells (e.g. neural stem cells). The treatment of these conditions such as HIVD in accordance with the present invention is particularly relevant due to the demonstrated effect of astrocytes on these conditions and in particular HIVD.

In accordance with the present invention, astroycte precursors such as APCs or IPAs are collected from a suitable source and homogeneous populations prepared using marker discrimination means as described above. At this point, the cells may be optionally frozen and stored for subsequent use. Alternatively, or after storage, the cells are expanded in vitro by the use of one or more growth factors and from about 10⁵ to about 10¹⁰ cells administered directly to the site affected on the brain or part thereof (e.g. retina) or other part of the CNS. Alternatively, or in addition, the cells may be administered to another part of the brain or CNS where they migrate to the site required since APCs and IPAs retain their migratory potential.

In vitro expansion is a particularly convenient form of expansion but as an alternative or in addition to in vitro expansion, the one or more growth factors may be administered to the brain or other part of the CNS to facilitate APCs or IPAs expanding in vivo.

Reference herein to “cell replacement therapy” includes, in one form, a process in which undifferentiated APCs and/or IPAs are strategically placed in vivo or in vitro such as to differentiate and proliferate into a mature form of astrocyte. Thus, cell replacement therapy requires that an undifferentiated astrocyte precursor cell appropriately differentiate for the purposes of providing repair, regeneration or replacement of a cell function. “Cell replacement therapy” also includes augmentation therapy. The latter includes the removal of existing cells or tissue, expanding in culture and then replacing. This is a particular advantage of the present invention, where a single or a few astrocyte precursor cells are capable of expansion in culture to give rise to a large number of astrocytes. The subject into which the purified astrocyte precursor cells are implanted for the purpose of “cell replacement therapy” or repair of tissue, or from which stem cells can be derived, is preferably an animal including but not limited to animals such as cows, pigs, horses, chickens, cats, dogs and is preferably a mammal such as a primate and most preferably a human.

Furthermore, the essence of this aspect of the present invention is the co-transplantation of APCs and/or IPAs with neural stem cells to enhance the proliferative, differentiative and/or maturation of both lineages. The latter includes neuronal stem cells. The former includes various growth factors. The term “co-introduction” or “co-introduced” includes the simultaneous or sequential administration of both the astrocytes and other cell or factor.

The present invention further contemplates using the astrocyte cell markers in a range of diagnostic applications in addition to using the markers to selectively isolate astrocytes at a particular level of maturity. For example, in accordance with the present invention it has been elucidated that Pax2 is no longer expressed or is only poorly expressed in a proportion of adult astrocytes and in a larger proportion of aged astrocytes. However, certain disease conditions or ageing may result in adult astrocytes beginning to express Pax2. The identification of Pax2 expression in adult astrocytes and aged astrocytes may be indicative of particular disease condition, neurological dysfunction, level of ageing or a propensity to develop any of the latter conditions.

Accordingly, the present invention contemplates a method for assessing the level of healthy tissue in a CNS biopsy such as a brain biopsy in an adult subject said method comprising determining in said biopsy presence of Pax2⁺ astrocyte cells wherein the presence of said Pax2⁺ cells is indicative of a reversion in the maturation of said astrocytes.

As indicated above, however, there are a range of markers which can be used to characterize the astrocyte cell populations.

The identification of new growth factors for APCs and IPAs is further contemplated by the present invention. In one embodiment, cells are cultured in vitro and the culture supernatant tested using, for example, microarray technology or the cells themselves tested for differential gene expression between different stages of maturation.

Suitable sources of astrocyte precursors include embryo or fetal brain including retinal tissue or other CNS tissue. As stated above, the isolated cells may be used immediately, subject to expansion in vitro and/or stored for subsequent use. The cells may be from the same subject being treated (autologous therapy) or a different subject (non-autologous therapy). Autologous therapy is preferred.

In one embodiment, therefore, there is provided a composition of astrocyte precursor cells such as APCs or IPAs in substantially homogeneous form, said composition optionally further comprising one or more pharmaceutically-acceptable carriers and/or diluents.

Methods for preparing cell compositions are well known in the art. The composition of cells may also be stored in vials or other convenient container means. The containers may also be in package form with instructions for use.

As stated above, the present invention extends to growth factors from astrocyte precursors.

These may be identified by any number of techniques including microarray technology. The latter is useful for identifying growth factor or autocrine factor receptors on the surface of particular astrocytes. This will then enable selection of particular growth or autocrine factors which will facilitate differential and/or proliferation of particular astrocyte cells.

Furthermore, differential hybridization is another useful technique for identifying other markers including growth or autocrine factor receptor markers or particular astrocyte cells.

Another aspect of the present invention is directed to conditioned medium from the in vitro culture of astrocyte precursor cells such as APCs or IPAs wherein said conditioned medium comprises one or more growth factors or autocrine factors.

More particularly, the present invention contemplates a growth or autocrine factor obtainable from conditioned medium of an in vitro cell culture of astrocyte precursors such as APCs or IPAs. Examples of growth and autocrine factors include CNTF, LIF, BMP, TGF-β and EGF.

In an alternative embodiment, the present invention provides a growth factor or autocrine factor identified by microarray technology of astrocyte cells.

The growth or autocrine factor may be in isolated form or may be in composition form such as comprising one or more pharmaceutically-acceptable carriers and/or diluents. This factor may be administered directly to the brain or retina or other suitable location to facilitate growth development of replacement tissue. This is particularly possible since, in accordance with the present invention, Pax2⁺ GFAP⁻ APCs and Pax2⁺ GFAP⁺ astroctyes have been identified in the sub-ventricular zone of the lateral ventricle of the adult brain. In contrast, no Pax2⁺ GFAP⁻ APCs have been identified in the sub-ventricular zone of the third ventricle where GFAP⁺ Pax2⁻ adult/aged astrocytes have been identified.

The present invention further contemplates the use of the purified astrocytes and in particular astrocyte precursor cells as gene therapy carriers. According to this embodiment, genetic material, encoding neurobiologically-useful factors, is introduced into the astrocytes prior to administration to a subject. Neurobiologically-useful factors contemplated by the present invention include growth factors, cytokines, proliferation and/or differentiation promoting agents and anti-viral and other anti-pathogenic agents. In accordance with this aspect of the invention, genetic material encoding these factors is cloned into a variety of vectors including viral vectors and introduced into the cultured astrocytes. After appropriate selection, including optionally stable integration of the genetic material into the chromosome of the astrocytes, these can then be used in tissue repair or augmentation therapy.

The present invention further provides a system of lineage-specific cell isolation from tissue of the CNS by the method of:—
(1) obtaining a sample from the CNS;
(2) separating the cells from the CNS on the basis of cell surface markers;
(3) selecting cells having desired cell surface markers and making these available for autologous or non-autologous transplantation; and
(4) optionally co-administering the isolated cells with neural cells, neural stem cells or mature astrocytes or autocrine factors.

The present invention is further described by the following non-limiting Examples.

### EXAMPLE 1

### Collection, Age Determination and Preparation of Human CNS Tissue

Human fetal eyes, ranging in age from 8 to 32 weeks gestation were used in accordance with the guidelines set forth in the Declaration of Helsinki. Fetuses older than 20 weeks gestation had died of natural causes after premature or difficult deliveries. Younger fetuses were obtained after water bag- or prostaglandin-induced abortions, which are permitted up to 20 weeks gestation. Embryonic or fetal brain tissue also provided a useful source of cells. The age of each fetus was determined from charts of crown-rump length and crown-heel length (Potter, E. L. and Graig, J. M., Pathology of the Fetus and the Infant, pp. 29-37, Yearbook Medical Publishers, Chicago, 1975). Three adult human retinas were obtained from an eye bank and originated from individuals aged 69, 69, and 79 years; the latter individual had a history of lung carcinoma whereas the other two had no significant medical history.

For preparation of retinal whole-mounts, the anterior segment and vitreous of each eye were removed and the eyecup was fixed with 4% w/v paraformaldehyde in 0.1 M sodium phosphate buffer (pH 7.4) for a minimum of 2 days at 4° C. The retina was dissected as previously described (27) and permeabilized by washing for 30 min in phosphate-buffered saline (PBS) containing 1% v/v Triton X-100. After blocking of nonspecific binding sites by incubation for 1 h with PBS containing 1% v/v bovine serum albumin, whole-mounts were exposed to primary antibodies.

For cryostat sections, the eyeball was washed several times in PBS and then incubated at 4° C. in 25% w/v sucrose overnight, embedded in OCT compound (Miles, Elkhart, Ind.), and rapidly frozen in isopentane-cooled liquid nitrogen. Cryostat sections (20 μm) were cut at −20° C. and mounted on gelatin-coated slides.

Whole-mounts of retinas from fetuses at various ages and from adults, as well as cryostat sections of fetal and adult eyes, were examined with various combinations of antibodies to identify APCs and perinatal astrocytes. The antibodies were as follows: (i) rabbit polyclonal antibodies to Pax2 (1:100 dilution) (Babco), which were generated in response to a recombinant protein containing amino acids 188 to 385 of Pax2 and which react with the Pax2 proteins of a variety of species, including mouse, rat, human, chicken, Xenopus, and zebrafish; (ii) mouse monoclonal antibody (mAb) GA5 to GFAP (IgG1; 1:100 dilution) (Sigma); and (iii) mouse mAbs 3B4 (IgG2A; 1:4 dilution) (Boehringer Mannheim) and LN-6 (IgM; 1:200 dilution) (Sigma) to vimentin. To determine the relation between Pax2 immunoreactivity and the developing vasculature, the inventor labeled some retinas with both anti-Pax2 and the mouse mAb QBEND/10 to CD34 (IgG1; 1:50 dilution) (Serotec). Retinas were also labeled with both anti-CD34 and rabbit polyclonal antibodies to GFAP (1:2 dilution) (Biogenex) to determine the relation between astrocyte differentiation and blood vessel formation, as previously described (Hughes et al., 2000, supra).

The glial population obtained following positive selection with markers such as A2B5, GD3, 3CB2, FGFR3 and PDGFRA are cultured in a serum free medium such as DMEM/F-12 together with growth factors. These growth factors include bFGF and chick embryo extract.

### EXAMPLE 2

### Pax2-GFAP, Pax2-vimentin, Pax2-CD34, and CD34-GFAP Double-Label Immunohistochemistry

Retinal whole-mount preparations were incubated for 2 to 3 days at 4° C. with a mixture of anti-Pax2 and either mouse anti-GFAP, anti-vimentin, or anti-CD34. They were then washed three times with PBS containing 0.1% v/v Triton X-100, incubated for 4 h with a mixture of Cy3-conjugated goat antibodies to rabbit IgG (1:200 dilution) (Jackson ImmunoResearch) and fluorescein isothiocyanate (FITC)-conjugated sheep antibodies to mouse Ig (1:50 dilution) (Amersham), and washed three times with PBS containing 0.1% v/v Triton X-100. For confocal microscopic analysis of double labeling with anti-CD34 and rabbit anti-GFAP, retinas were incubated overnight at 4° C. with the primary antibodies, washed and then incubated with appropriate secondary antibodies as described above. For light microscopy, retinas were labeled with polyclonal anti-GFAP as described previously (Hughes et al., 2000, supra) and then with anti-CD34. Labeling with anti-CD34 was detected by incubation at room temperature first for 4 h with biotinylated secondary antibodies (1:50 dilution) (Amersham) and then for 2 h with alkaline phosphatase-conjugated Extravidin (1:100 dilution) (Sigma); the alkaline phosphatase reaction was performed with Fast Red tablets (Sigma). Whole-mounts were mounted in glycerol-PBS (1:2) with the nerve fiber layer (NFL) uppermost, with the exception that, for examination of the ventricular zone, they were mounted with the retinal pigment epithelium (RPE) uppermost.

Cryostat sections were allowed to air-dry for 15 min, after which non-specific binding sites were blocked by incubation for 30 min with PBS containing 1% v/v bovine serum albumin. Sections were then incubated overnight at 4° C. in a humidified atmosphere with various combinations of primary antibodies, washed three times with PBS, and incubated for 2 h with appropriate secondary antibodies as described above. The sections were finally washed several times with PBS and mounted in glycerol-PBS (1:2).

### EXAMPLE 3

### Triple-Label Immunohistochemistry for Pax2, Vimentin and GFAP

For triple labeling, retinal whole-mounts and sections were incubated overnight at 4° C. in a humidified atmosphere with a mixture of anti-Pax2 and anti-vimentin. After washing three times with PBS, they were incubated for 2 h with Cy3-conjugated anti-rabbit IgG and either FITC-conjugated goat anti-mouse IgG2a (1:50 dilution) (Southern Biotechnology Associates) or Texas red-conjugated goat anti-mouse IgM (1:50 dilution) (Vector). The tissue was washed three times with PBS, incubated overnight at 4° C. with mouse anti-GFAP, washed three times with PBS, and incubated for 4 h with biotinylated goat anti-mouse IgG1 (1:50 dilution) (Southern Biotechnology Associates). After washing three times with PBS, the tissue was incubated overnight at 4° C. with AMCA-conjugated streptavidin (1:100 dilution) (MDA Pharma) or Cy5-conjugated streptavidin (1:100 dilution) (Jackson ImmunoResearch), washed with PBS, and mounted in glycerol-PBS (1:2).

### EXAMPLE 4

### Confocal Microscopy

Confocal microscopy was performed with a Leica argon-krypton laser mounted on a Leica Axiophot epifluorescence photomicroscope. FITC, Cy3, and Cy5 fluorescence was excited at 488, 588, and 665 nm, respectively. Images were collected at a resolution of 300 pixels per inch and processed with Adobe Photoshop V5.0 and Adobe PageMaker V6.5 software.

### EXAMPLE 5

### Mapping and Determination of Retinal Area and Cell Density

The outer limits of immunoreactivity and retinal outlines were determined with a Leica fluorescence microscope (model AH BT, attachment model AH2-RFL) with a 10× eyepiece containing a 1-mm grid (Chan-Ling et al., Current Eye Res. 9: 459-478, 1990; Chang Ling, T., Microsc. Res. Techniq. 36: 1-16, 1997). The maps were scanned with an XRS-OmniMedia -3cx flatbed scanner and processed with the use of Adobe Photoshop V5.0 software.

### EXAMPLE 6

### Specificity of Pax2 Expression to Astrocytic Lineage Cells in the Developing Human Retina

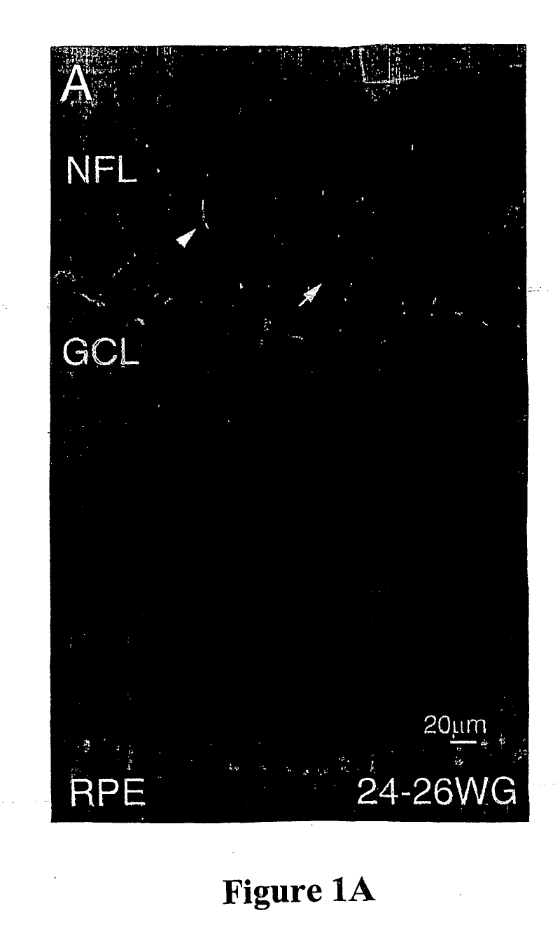

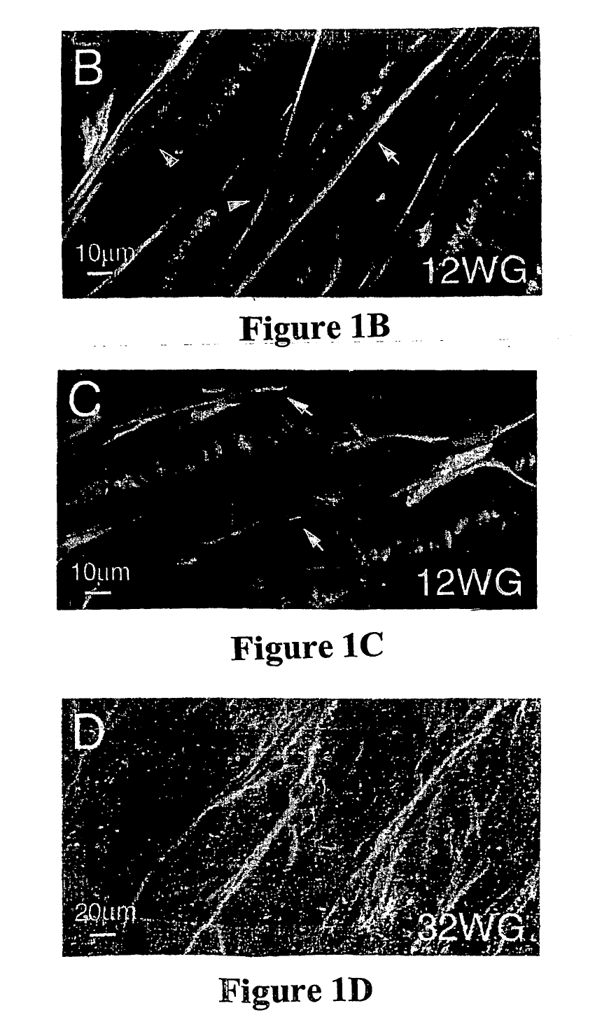

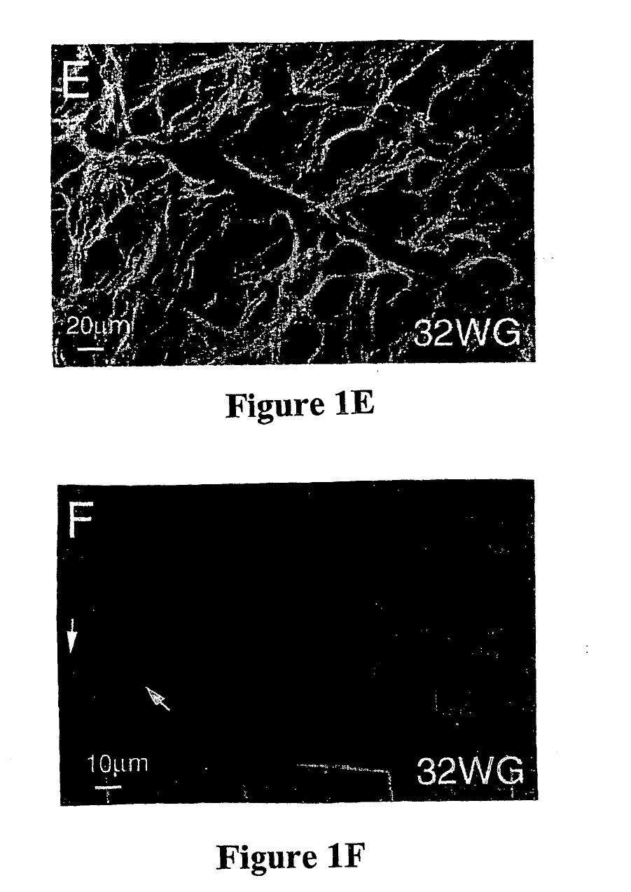

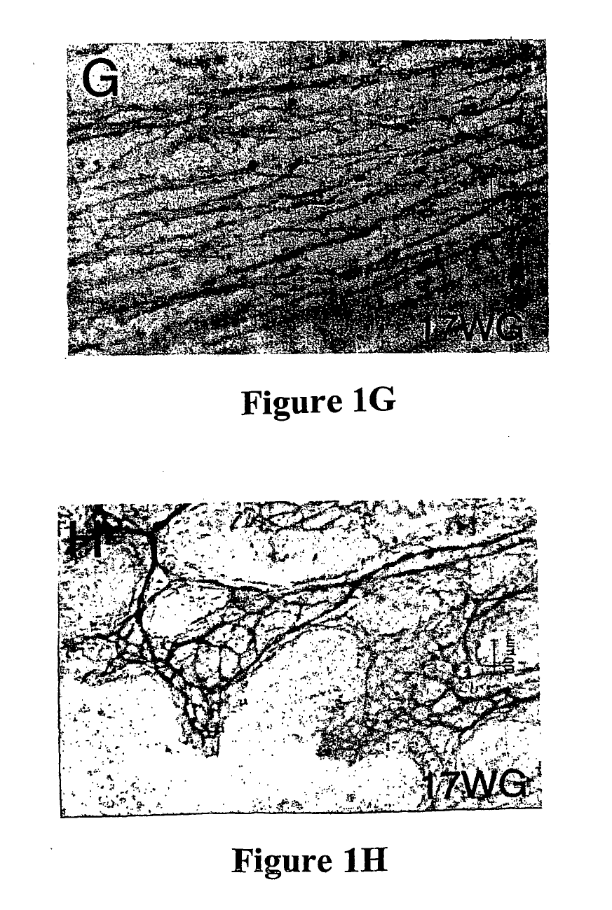

With the exception of a small region at the ventricular zone surrounding the ONH (see below), Pax2 expression was restricted to somas in the GCL and NFL of the developing human retina, as revealed by the retina at 24 to 26 weeks gestation shown in FIG. 1A. Triple-label immunohistochemistry with retinal whole-mounts showed that anti-Pax2 labeled only cells that were positive for the astrocytic lineage markers vimentin or GFAP (FIGS. 1, B and C). Double labeling with anti-CD34 and anti-Pax2 revealed that Pax2 is not expressed by endothelial cells. Three populations of Pax2⁺ cells were identified in the developing retina: (i) cells that were Pax2⁺, GFAP⁻, and vimentin⁺ (FIGS. 1, B and C) were designated APCs; (ii) cells that were Pax2⁺, GFAP⁺, and vimentin⁺ (FIG. 1B) were designated immature perinatal astrocytes; at 12 weeks gestation, most GFAP⁺ cells were vimentin⁺; and (iii) cells that were Pax2⁺, GFAP⁺, and vimentin⁻ (FIG. 1, D through F) were designated mature perinatal astrocytes; at 32 weeks gestation, most GFAP⁺ astrocytes were vimentin⁻. Thus, the transition from an APC to an immature perinatal astrocyte in vivo is characterized by the onset of expression of GFAP, and the transition from immature to mature perinatal astrocytes is characterized by the loss of expression of vimentin.

To distinguish astrocytes of the fetal and neonatal human retina from those present in the adult CNS, the inventor has adopted the term perinatal astrocytes, as used by Mi and Barres (1999, supra), throughout this study, even though these cells are already present in the embryonic retina. A similar terminology has been applied to cells of the oligodendrocyte lineage (Wolswijk, G. and Nobel, M., Development 105: 387-400, 1989), in which perinatal and adult oligodendrocyte precursor cells exhibit differences in such characteristics as cell cycle time, proliferative capacity and rate of migration.

A summary of markers expressed during the different developmental stages of the astrocytes is shown in Table 1.

### EXAMPLE 7

### Morphology and Location of APCs and Perinatal Astrocytes: Relations with Ganglion Cell Axons and Forming Blood Vessels

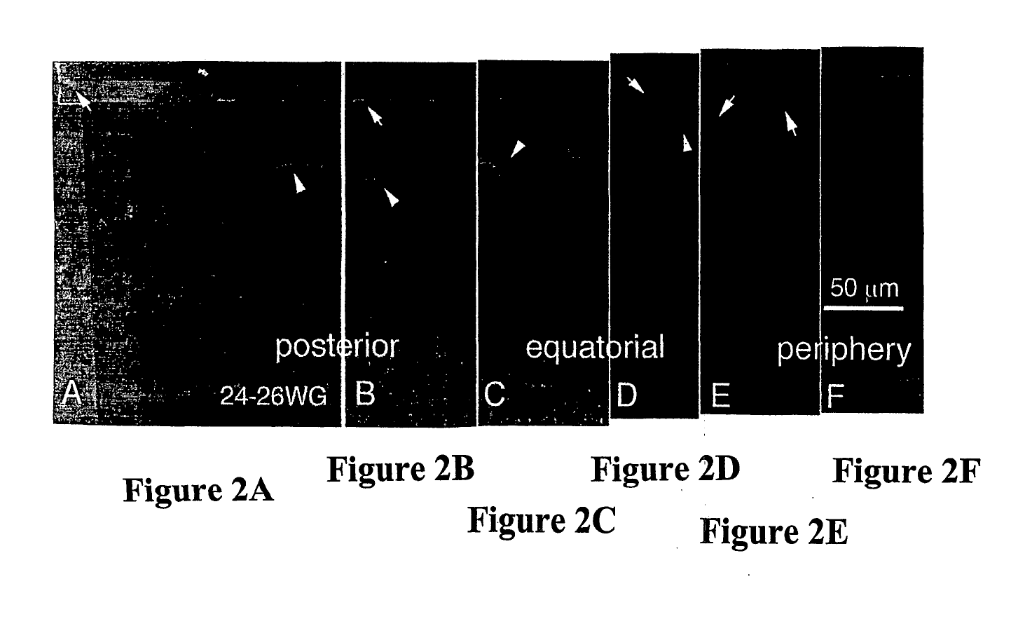

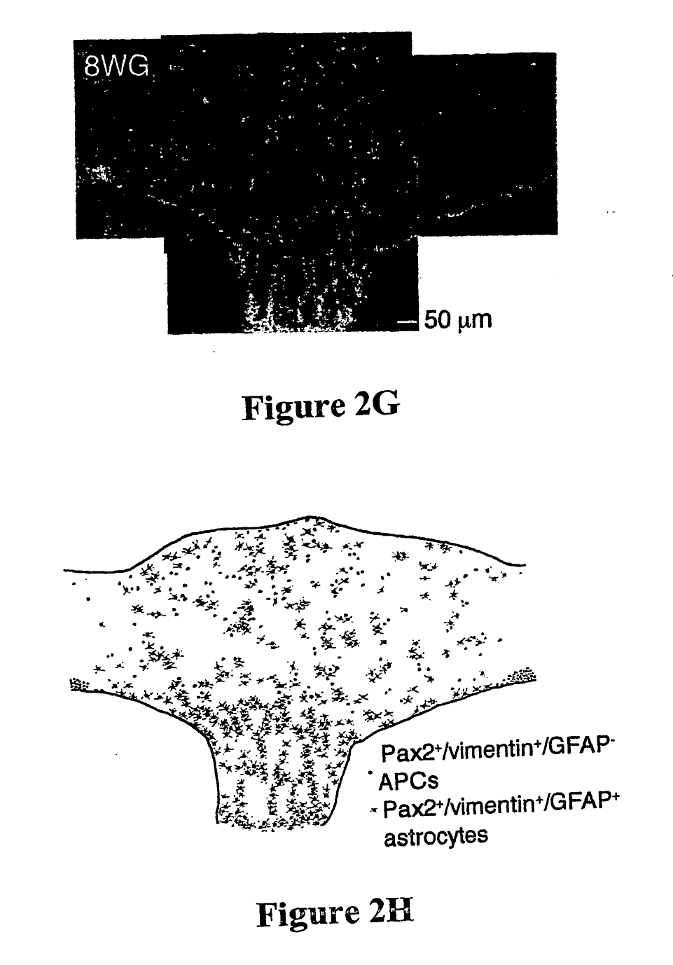

Pax2⁺, GFAP⁻ APCs were located superficially, adjacent to the inner limiting membrane, and were characterized by a predominantly spherical or ovoid morphology with a soma diameter of ˜12 to 25 μm (arrows in FIG. 1, A through C). APCs migrated superficially over regions of the retina containing immature perinatal astrocytes (arrows in FIG. 2, A, B, and D). At 24 to 26 weeks gestation, perinatal astrocytes were abundant in the central region of the retina (FIG. 2, A through C), whereas only Pax2⁺, GFAP⁻ APCs were evident more peripherally (FIG. 2, D and E). At the edge of the retina, no Pax2⁺ cells were evident at this time (FIG. 2F). Most immature perinatal astrocytes at the leading edge of GFAP immunoreactivity exhibited an approximately spherical soma, with a diameter of 6 to 10 μm, and possessed bipolar GFAP⁺ processes located superficially in the nerve fiber layer (NFL) (FIG. 1D). Mature perinatal astrocytes located more centrally in the NFL adopted a morphology characterized by multiple parallel processes and were closely aligned along nerve fiber bundles (NFBs) (FIG. 1G), whereas those located in the deeper ganglion cell layer (GCL) adopted a predominantly stellate morphology with numerous processes ensheathing blood vessels (FIG. 1E). Mature perinatal astrocytes exhibited an ovoid soma with a diameter of 6 to 10 μm. Consistent with previous observations (Provis, 1997, supra; Hughes et al., 2000, supra), the outer limit of astrocyte migration into the human retina preceded the outer limit of patent vessels visualized by anti-CD34 immunohistochemistry (FIG. 1H).

### EXAMPLE 8

### APCs and Perinatal Astrocyte Differentiation at the ONH

Retinal astrocytes immigrate from the optic nerve (Ling and Stone, 1988, supra; Watanabe, T. and Raff, M. C., Nature 332: 834-837, 1988; Ling et al., 1989, supra; Huxlin et al., J Neurocytol. 21: 530-544, 1992). The pathway of invasion of cells of the astrocytic lineage is, therefore, thought to lead from the ONH into the peripheral retina. The inventor applied double-label immunohistochemistry for Pax2 and GFAP to cryostat sections of eyes at 8, 12, 16, and 24 to 26 weeks gestation in order to examine the distribution of APCs and perinatal astrocytes at the ONH during development. Consistent with the previous demonstration of Pax2 gene expression in the region of the human optic disk and optic nerve (Terzic et al., 1998, supra), the inventor detected Pax2 immunoreactivity in the optic nerve and at the ONH at 8 weeks gestation (FIG. 2, G and H). Quantitative analysis of such a double-labeled section of the human ONH at 8 weeks gestation revealed that 34% of Pax2⁺ cells were Pax2⁺, GFAP⁻ APCs, with the remainder being Pax2⁺, GFAP⁺ perinatal astrocytes.

FIG. 2G is a photographic montage of the ONH region at 8 weeks gestation and FIG. 2H is a schematic representation of this region showing the precise locations of individual APCs and perinatal astrocytes. GFAP⁺ perinatal astrocytes had reached the distal limit of the human optic nerve, and many of these cells had extended into the ONH. APCs were no longer evident at the retinal end of the optic nerve, but were dispersed throughout the ONH.

### EXAMPLE 9

### APCs and Perinatal Astrocytes at the Ventricular Zone of the Fetal Retina

Transverse sections revealed a cluster of Pax2⁺ somas present in a small region surrounding the ONH at the ventricular surface of the human retina (FIG. 2, G through J). This cluster of Pax2⁺ somas was located at the innermost margin of the ventricular zone of the retina, at its junction with the numerous optic nerve axons that exit the retina to form the optic nerve. The presence of these cells and the extent of their distribution were conformed by examining the ventricular surface of retinal whole-mounts at various ages.

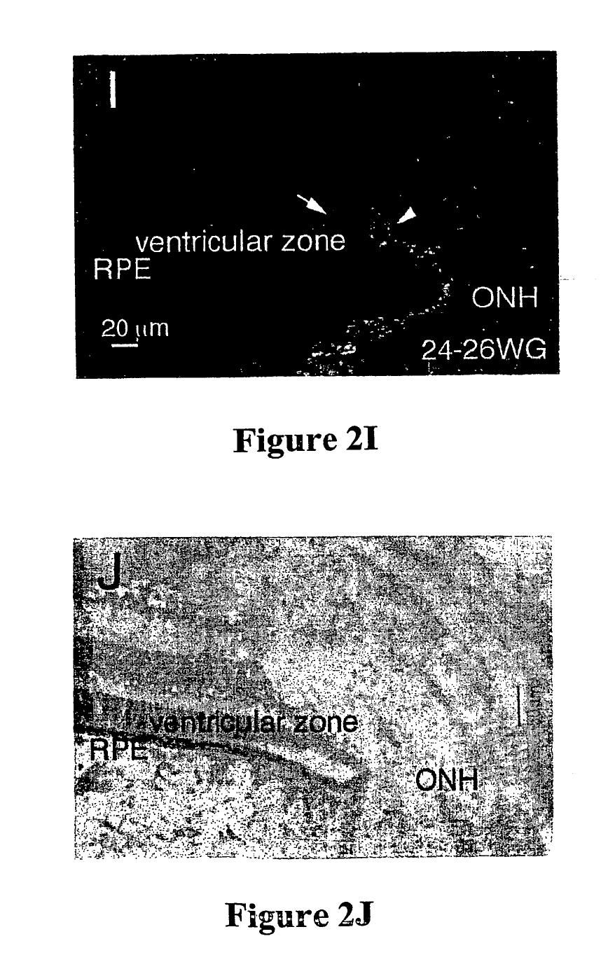

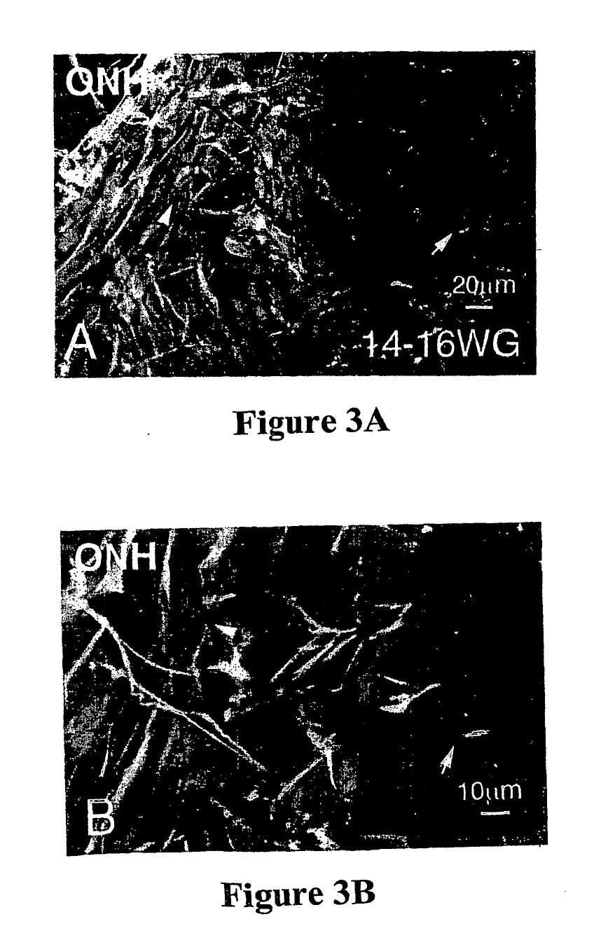

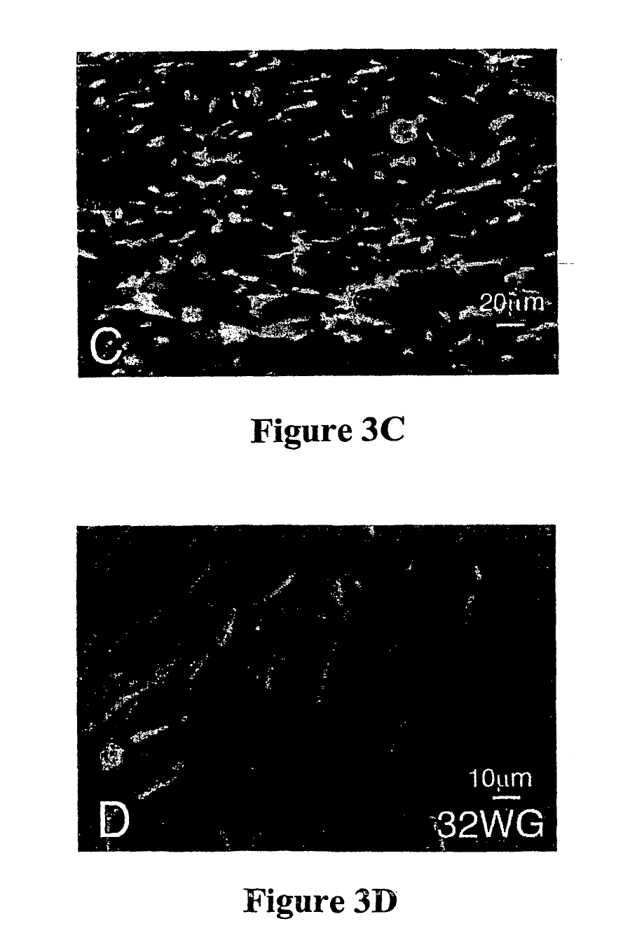

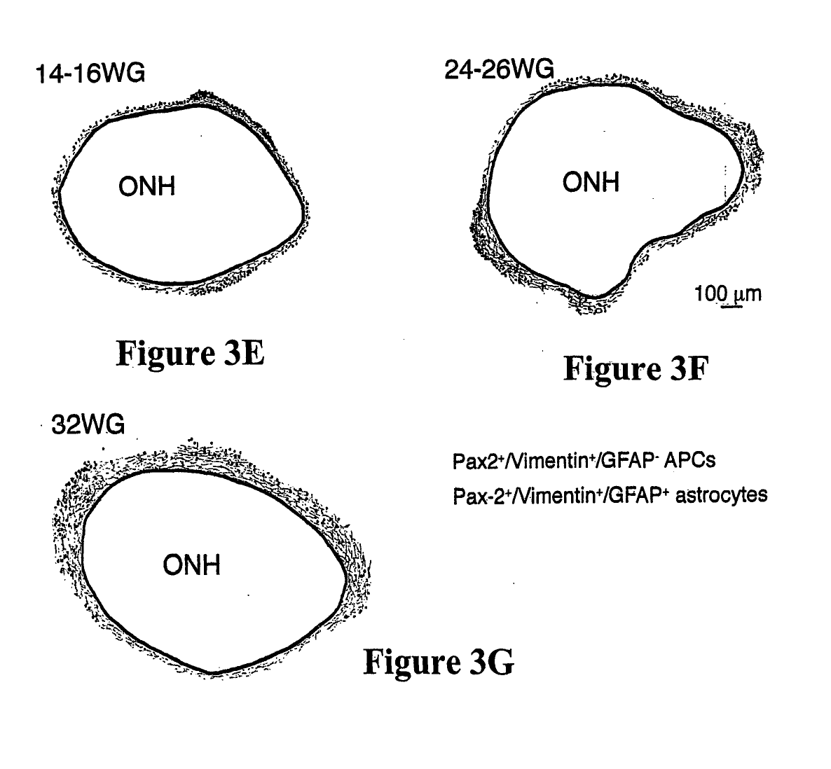

The morphology of these cells in the peripapillary region of the retina at 14 to 16 weeks gestation and 32 weeks gestation is shown in FIG. 3 (A through D). Two populations of Pax2⁺ cells were discerned at the ventricular zone: Pax2⁺, vimentin⁺, GFAP⁺ immature perinatal astrocytes located in the region surrounding the ONH, and Pax2⁺, vimentin⁺, GFAP APCs located predominantly at the periphery of the Pax2⁺ region. The extent of the distribution of perinatal astrocytes as well as the precise locations of individual Pax2⁺ APCs at the ventricular surface of the retina at various fetal ages are shown in FIG. 3 (E through G).

### EXAMPLE 10

### Timing and Topography of APC and Perinatal Astrocyte Invasion of the Retina

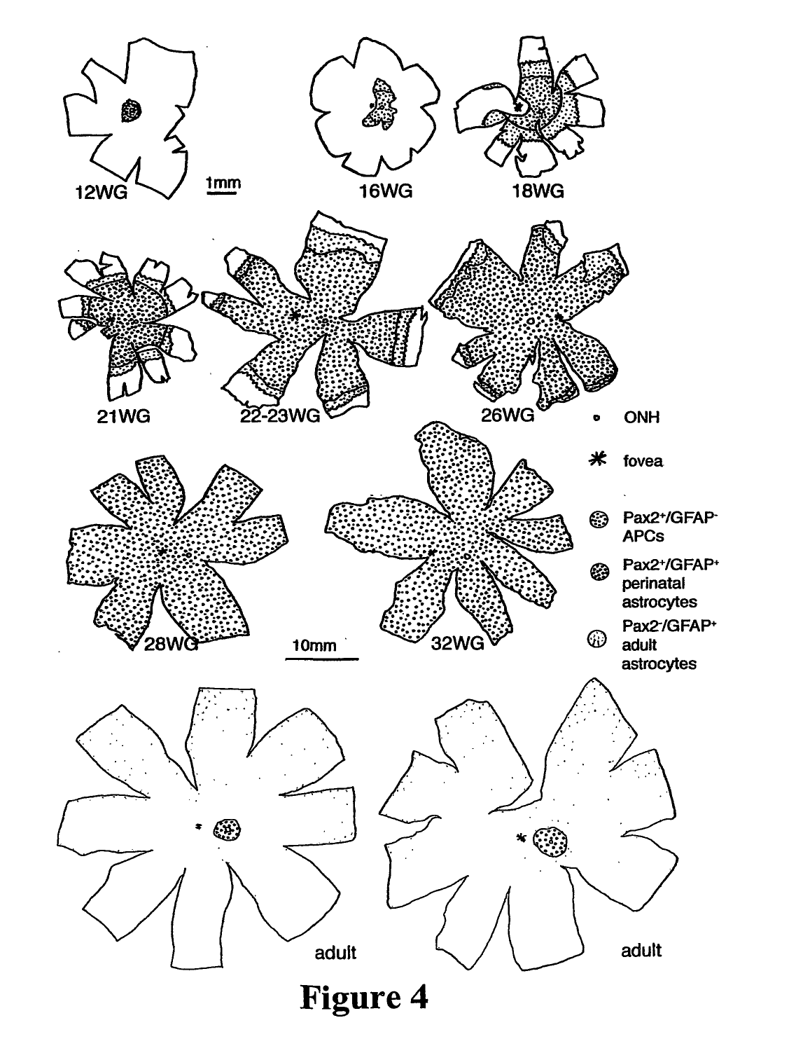

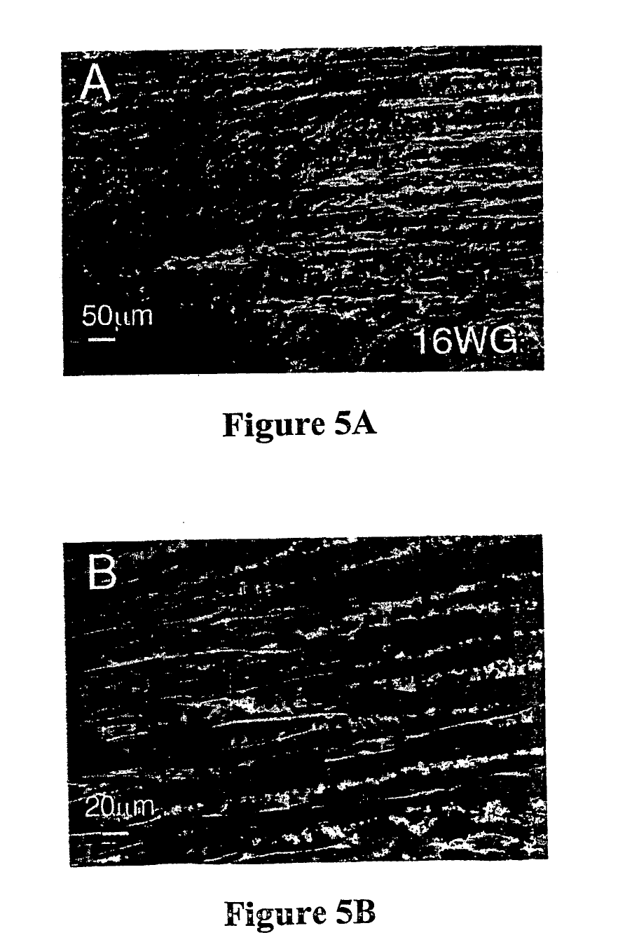

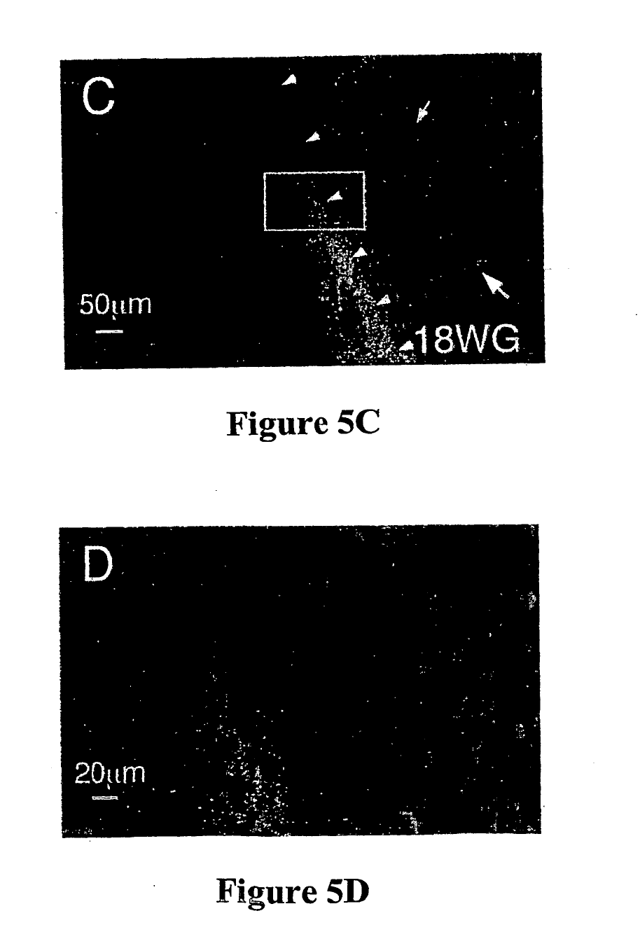

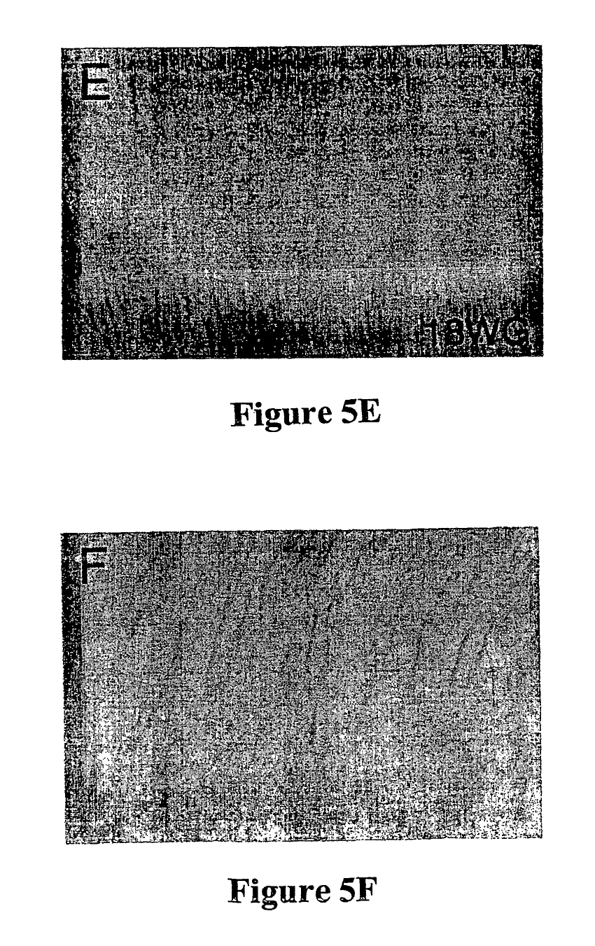

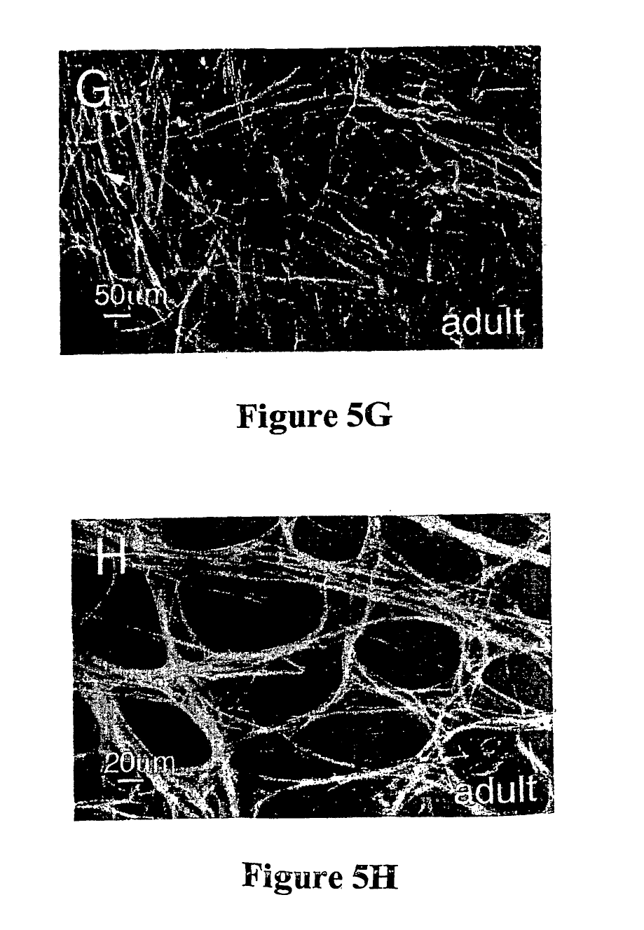

Pax2⁺, GFAP⁻ APCs were consistently detected in a small region ahead of the Pax2⁺, GFAP⁺ perinatal astrocytes during development of the human retina (FIG. 4). Triple labeling confirmed that all cells at the leading edge were Pax2⁺, vimentin⁺, GFAP⁻ APCs (FIG. 5, A and B). Even at the youngest age examined with transverse sections (8 weeks gestation), APCs had extended ˜560 μm from the ONH, and, with increasing maturation, the outer limit of their distribution expanded (FIG. 4). At each stage of maturation, the outer limit of perinatal astrocytes lagged behind that of APCs. The spread of both APCs and perinatal astrocytes was centered on the ONH, and it showed an approximately four-lobed topography early in embryonic development (FIG. 4). APCs and differentiated astrocytes reached the edge of the retina by 28 weeks gestation and between 28 and 32 weeks gestation, respectively. APCs persisted at reduced densities throughout the retina at 32 weeks gestation, the oldest fetal age examined.

APCs and perinatal astrocytes followed a curved pattern of migration in the temporal retina, mimicking the pattern of NFBs and blood vessels in the human retina. The density of APCs and perinatal astrocytes was markedly reduced in the raphe region. Throughout the observation period, neither APCs nor committed astrocytes were detected in the incipient foveal zone (FIG. 4; FIG. 5, C through E), consistent with previous observations (Ramirez et al., 1994, supra; Trivino et al., 1997, supra). However, individual isolated astrocytes were apparent in the perifoveal region at 18 weeks gestation (FIG. 5, C and D), and, at 18 weeks gestation, perinatal astrocytes were observed aligned along NFBs in the raphe region (FIG. 5, E and F).

### EXAMPLE 11

### Astrocytic Lineage in the Adult Human Retina

The inventor's observation that APCs are present in substantial numbers in the human retina at 32 weeks gestation prompted us to examine whether such cells also exist in the adult retina. The inventor applied triple-label (Pax2-GFAP-vimentin) immunohistochemistry to retinal whole-mounts and transverse sections prepared from three adult human eyes. Anti-Pax2 did not label neurons or endothelial cells in the adult human retina, and GFAP⁺ astrocytes showed a morphology and distribution consistent with those previously described (Ramirez et al., 1994, supra; Trivino et al, 1997, supra). The inventor identified two distinct populations of astrocytic cells in the adult human retina: Pax2⁺, GFAP⁺, vimentin mature perinatal astrocytes (FIG. 5G) and Pax2⁻, GFAP⁺, vimentin⁻ adult astrocytes (FIG. 5H).

Topographical analysis of the distribution of these two types of astrocytes revealed that Pax2⁺, GFAP⁺, vimentin⁻ mature perinatal astrocytes were restricted to the region surrounding the ONH, whereas Pax2⁻, GFAP⁺, vimentin adult astrocytes were present throughout the retina with the exception of the foveal region (FIG. 4; FIG. 5, G and H). Thus, in the adult human retina, most cells of the astrocytic lineage no longer express Pax2. Because of the ability to detect small numbers of cells with the retinal whole-mount technique (Hu et al., Am. J. Pathol. 156: 1139-1149, 2000), the inventor mapped two whole-mounts to determine whether any APCs were present in the normal adult human retina. Every Pax2⁺ soma was checked for surrounding GFAP immunoreactivity, and all were found to be GFAP⁺. On the basis of this systematic evaluation, it is concluded that no Pax 2⁺, GFAP⁻ APCs were present in the adult human retinas, which were derived from individuals over 65 years of age.

Although the inventor detected both APCs and committed astrocytes at the ventricular zone in the region adjacent to the ONH during embryonic development, triple-label immunohistochemical analysis of transverse sections failed to detect either of these cell types in this region of the adult human retina.

### EXAMPLE 12

### Implications for Optic Nerve Coloboma

The observation that Pax2 expression is specific to cells of the astrocytic lineage in the intact human fetal retina suggests that congenital optic nerve colobomas might be attributable to aberrant astrocytic differentiation in the ventricular zone during embryonic development. Colobomas result from imperfect formation or closure of the fetal cleft of the optic vesicle during embryogenesis.

Some human optic nerve colobomas are associated with abnormalities in Pax2 gene expression (Sanyanusin et al., 1995, supra). In normal human embryos, Pax2 mRNA is abundant in the optic nerve and ONH during the period of expected closure of the choroidal fissure (Terzic et al., 1998, supra). In mice with impaired expression of Pax2, the band of Pax2⁺ cells that surrounds the retinal ganglion cell axons as they exit the retina becomes disorganized and the surrounding retinal tissue is no longer clearly separated from the axons, resulting in dispersal of the axons over a much wider region (Otteson et al., 1998, supra). The inventor has now shown that the Pax2⁺ cells of this cuff in the human retina comprise APCs and perinatal astrocytes, suggesting that Pax2⁺ cells of the astrocytic lineage play a critical role both in delineating the axons of the ONH from the surrounding retinal tissue during development and in funneling and restricting the pathway of axonal exit from the retina. Thus, optic nerve colobomas may be caused by aberrant differentiation of cells of the astrocytic lineage at the peripapillary zone.

### EXAMPLE 13

### In Vivo Characterization of Type-1 APCs in Rat Retina

A developmental series of rat retinal whole-mount preparations from E15 to adulthood is subjected to double-label IHC for Pax2 and GFAP. Type-1 APCs is defined by the phenotype Pax2⁺ GFAP⁻, whereas committed astrocytes is defined by the phenotype Pax2⁺ GFAP⁺. The temporal and topographical distributions of APCs and committed astrocytes are determined, as are the morphology of these cells and their associations with neighbouring structures such as neurons and blood vessels. Animals are anaesthetized with sodium pentobarbitone (60 mg/kg, i.p.) and perfused transcardially with phosphate-buffered saline containing 4% v/v paraformaldehyde. Retinal whole-mount preparations and transverse sections are prepared as previously described.

Preliminary observations by the inventor have shown that APCs are present in the central rat retina for only a limited period (E16 to E20). Labeling with bromodeoxyuridine is also combined with Pax2 or GFAP IHC to determine the proliferative capacity of APCs and committed astrocytes in the retina at various stages of development. These studies provide important information on the morphology, antigenic phenotype, migration, and proliferation of astrocytes at various stages of differentiation in vivo. Various astrocyte-specific markers, including vimentin and S100, as well as vascular markers are also examined as controls.

### EXAMPLE 14

### Isolation, Purification, Expansion and Controlled Differentiation of APCs from Embryonic Rat Retina

Regions of the central rat retina in which APCs are determined to be abundant are isolated from embryos on E17 to E18, the stage of development at which the preliminary data indicate that the number of APCs is maximal. The tissue is dissociated according to standard protocols and APCs are isolated by positive or negative immunopanning or fluorescence-activated cell sorting with the use of antibodies to specific cell epitopes. Markers appropriate for negative selection of vascular endothelial cells, microglia, oligodendrocytes and their precursors and neurons include the Griffonia simplicifolia (GS) lectin, C5, embryonic N-CAM, and the ED1 and 04 antibodies. Immunohistochemistry will reveal a substantial population of cells which are Pax2⁺ and GD3⁺ among a predominantly GFAP⁻ population, indicating that GD3 is an appropriate surface marker for APCs. Other positive selection markers include A2B5, 3CB2, PDGFRα and FGFR3. Purified populations of APCs are cultured in plates coated with either fibronectin alone or fibronectin plus laminin, and maintained in SATO defined medium supplemented with various growth factors both to facilitate their expansion and to inhibit their differentiation into committed astrocytes. Growth factors tested alone and in combination include CEE, basic fibroblast growth factor, neurotrophin-3 and ciliary neurotrophic factor. Various conditions, including withdrawal of CEE and exposure to thyroid hormone, retinoic acid, ciliary neurotrophic factor, leukemia inhibitory factor, or bone morphogenetic proteins, are also tested for their ability to induce differentiation of APCs.

### EXAMPLE 15

### Analysis of Gene Expression in Cells of the Astrocyte Lineage

The major obstacle of differential display is the issue of discriminating between false positives and truly differentially expressed mRNAs. This process is arduous and requires large amounts of RNA. To overcome this problem, cDNA probes are generated from amplified RNA following standard protocols that allow differential screening of mRNA species with a frequency as low as 1/40,000. RNA is extracted from pure, expanded populations of type-1 APCs and linearly amplified an estimated 10⁶-fold using T7 RNA polymerase amplification (Ampliscribe T7 Transcription Kit, Epicentre Technologies, Madison, Wis.). Cy3-labeled cDNA synthesized from the amplified RNA is hybridized to rodent microarrays and analyzed using ScanArray 3000 reader (General Scanning, Watertown, Mass.). The pattern of gene expression is determined for the retinal APCs are compared with those of neuroepithelial stem cells, GRP cells, and neuron-restricted precursor cells of the spinal cord as well as O-2A progenitor cells of rat optic nerve. The pattern of gene expression is thus examined to identify Type I astrocyte-specific genes at various stages of differentiation.

### EXAMPLE 16

### Determination of the Developmental Potential of APCs In Vitro and In Vivo

APCs is subjected to clonal analysis by plating at a density of 10 to 50 cells per dish. Single cells are identified after culture for 4 h, expanded in medium supplemented with appropriate growth factors for 4 days, and induced to differentiate. Clonal plates are then subjected to triple-label cytochemical analysis of appropriate cell markers in order to identify the progeny of the precursor cells. Cells at various stages of differentiation are thus identified by their expression of developmental stage-specific markers.

In addition to these in vitro experiments, pure populations of APCs labeled with green fluorescent protein are injected into the vitreous chamber of adult rats or rat pups during the first week after birth with the use of a 10-μl Hamilton-type microsyringe. Retroviral vectors and virus preparation are then carried out. All studies are undertaken with inbred Fischer 344 rats to allow syngeneic transplantation. Cultured APCs will be harvested with trypsin, washed and suspended at a density of 100,000 cells with media containing 20 ng of bFGF. Initially, 10,000 cells will be transplanted per eye in order to assess the survival and incorporation into the host tissue of the donor cells, which will be examined after 21 days.

### EXAMPLE 17

### DNA Microarray

DNA microarray technology is used to identify markers and growth factor receptors that are expressed at various stages of the astrocyte lineage during development. Such information is fundamental to understanding the developmental biology of these cells and facilitates isolation and expansion of cell populations suitable for clinical application in the repair of degenerative neuropathies. In addition, characterization of conditions that promote expansion of APCs renders feasible the recruitment of endogenous populations of these cells for tissue repair.

### EXAMPLE 18

### In Vivo Model

Populations of rat APCs labeled with GFP are implanted into the retina of neonatal and adult rats and the fate of these cells examined in order to assess their potential for integration into the normal developing and adult CNS. Studies designed to determine whether co-transplantation of rat APCs with neural precursor cells facilitates neural repair by the latter cells in various experimental models of CNS damage are conducted including the rat retina injured by transient ischaemia and the retina of immature and mature dystrophic rats. Further, similar studies will be undertaken in the rat model of foetal alcohol syndrome. These various studies provide a source of human cells available to neurosurgeons for transplantation in humans.

### EXAMPLE 19

### Characterization of Cells of the Astrocyte Lineage in Human Fetal, Adult and Aged Brains In Vivo

Sections of human brain derived from embryos and adults of various ages are subjected to triple-label immunohistochemical analysis with antibodies to Pax2, vimentin and GFAP, according to the protocols described above in order to characterize the antigenic phenotype, timing of differentiation, distribution and morphology of cells of the astrocytic lineage.

These studies complement the characterization of this cell lineage in the human and rat retina (fetal, adult and aged) and provide important information on the morphology, antigenic phenotype and distribution of cells of the astrocytic lineage. Studies on human fetal brains define the developmental period during which APCs can be isolated from the human fetal brain as well as the regions of the brain that provide the highest yield of these cells. Furthermore, they will shed light on the regions of the normal adult human brain where APCs persist. Preliminary observations during physiological aging of the human brain have demonstrated a marked regional and individual variation in the distribution of the APC in adult human brain. In particular, Pax2⁺ GFAP⁻ APCs and Pax2⁺ GFAP⁺ astroctyes have been identified in the sub-ventricular zone of the lateral ventricle of the adult brain. In contrast, no Pax2⁺ GFAP⁻ APCs have been identified in the sub-ventricular zone of the third ventricle where GFAP⁺ Pax2⁻ adult/aged astrocytes have been identified.

### EXAMPLE 20

### Isolation and Purification of APCs from Human Fetal Brain and Retina and Determination of Culture Conditions that Support the Expansion and Controlled Differentiation of these Cells In Vitro

Human brain and retinal tissue are isolated from foetuses at the stage of development at which the number of these cells is maximal as determined by studies of intact fetal brain and retina.

### EXAMPLE 21

### Analysis of the Pattern of Gene Expression and the Developmental Potential In Vitro of Human APCs

The pattern of gene expression is examined in human APCs with the use of DNA microarray analysis. The developmental potential of these cells is determined by clonal analysis in vitro.

### EXAMPLE 22

### Determination of the Ability of Rat APCs to Facilitate the Migration, Integration and Differentiation of Neural Precursor Cells when Co-Transplanted with these Latter Cells in Various Experimental Models of Neurodegenerative Conditions in which Astrocytes Play a Roly in Pathophysiology

As groundwork for clinical trials of co-transplantation of APCs and stem cells and/or neural precursor cells, studies are undertaken aimed at characterizing the effects of implantation of rat APCs and stem cells and/or neural precursor cells in various experimental models of CNS trauma and disease.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

1. A method for generating a substantially homogeneous population of lineage-specific cells from tissue of the central nervous system (CNS) of mammalian animals, said method comprising subjecting said CNS tissue to tissue disruptive means to provide a mixed population of cells comprising the lineage-specific cells to be isolated, subjecting the mixed population to cell separation discrimination means to generate a substantially homogeneous population of lineage-specific cells.

2. The method of claim 1 wherein the lineage-specific cells are astrocyte precursor cells (APCs).

3. The method of claim 1 wherein the lineage-specific cells are immature perinatal astrocytes (IPAs).

4. The method of claim 1 wherein the lineage-specific cells are mature perinatal astrocytes (MPAs).

5. The method of claim 1 wherein the lineage-specific cells are adult or aged astrocytes.

6. The method of claim 1 wherein the mammalian animal is at a prenatal stage.

7. The method of claim 1 wherein the mammalian animal is at a postnatal stage.

8. The method of claim 7 wherein the postnatal animal is an adult.

9. The method of claim 1 wherein the separation discrimination means is based on a different range of cell markers present at different developmental stages of the lineage-specific cells.

10. The method of claim 9 wherein the separation discrimination means is based on positive or negative selection of cell surface markers.

11. The method of claim 10 wherein the neuronal cells are removed by negative selection with N-CAM.

12. The method of claim 10 or 11 wherein glial cells are selected positively using one or more of A2B5, GD3, 3CB2, FGR3, PDGFRα or a combination thereof.

13. The method of claim 10 or 11 or 12 wherein oligodendrocytes are removed by negative selection with one or more of Gal-C, 01, 04, anti-Mog or NG2 or a combination thereof.

14. The method of claim 9 wherein the cells are sorted by recognition of cell markers by immunological reagents.

15. The method of claim 10 wherein the immunoglobulin reagents are antibodies.

16. The method of claim 2 wherein the cells isolated are immunohistochemically Pax2⁺ GFAP⁻.

17. The method of claim 2 wherein the cells isolated are immunohistochemically Pax2⁺ GFAP⁺.

18. The method of any one of claims 1 to 17 wherein the mammalian animal is a human.

19. The method of any one of claims 1 to 17 wherein the mammalian animal is a livestock animal, laboratory test animal or a companion animal.

20. A substantially homogeneous population of mammalian lineage-specific cells from the CNS, said cells made by the method comprising subjecting said CNS tissue to tissue disruptive means to provide a mixed population of cells comprising the lineage-specific cells to be isolated, subjecting the mixed population to cell separation discrimination means to generate a substantially homogeneous population of lineage-specific cells.

21. The population of mammalian cells of claim 20 wherein the lineage-specific cells are astrocyte precursor cells (APCs).

22. The method of claim 20 wherein the lineage-specific cells are immature perinatal astrocytes (IPAs).

23. The method of claim 20 wherein the lineage-specific cells are mature perinatal astrocytes (MPAs).

24. The method of claim 20 wherein the lineage-specific cells are adult or agedd astrocytes.

25. The method of claim 20 wherein the mammalian animal is at a prenatal stage.

26. The method of claim 20 wherein the mammalian animal is at a postnatal stage.

27. The method of claim 26 wherein the postnatal animal is an adult or aged astrocyte.

28. The method of claim 20 wherein the separation discrimination means is based on a different range of cell markers present at different developmental stages of the lineage-specific cells.

29. The method of claim 28 wherein the separation discrimination means is based on positive or negative selection of cell surface markers.

30. The method of claim 29 wherein the neuronal cells are removed by negative selection with N-CAM.

31. The method of claim 28 or 29 or 30 wherein glial cells are selected positively using one or more of A2B5, GD3, 3CB2, FGR3, PDGFRα or a combination thereof.

32. The method of claim 28 or 29 or 30 or 31 wherein oligodendrocytes are removed by negative selection with one or more of Gal-C, 01, 04, anti-Mog or NG2 or a combination thereof.

33. The method of claim 28 wherein the cells are sorted by recognition of cell markers by immunological reagents.

34. The method of claim 33 wherein the immunoglobulin reagents are antibodies.

35. The method of claim 20 wherein APCs are isolated by the immunological separation of a population of Pax2⁺ cells followed by removal of GFAP⁺ cells form the Pax2⁺ population.

36. The method of claim 20 wherein IPAs are isolated by the immunological separation of a population of vimentin⁺ cells and then isolating GFAP⁺ cells from said vimentin⁺ population.

37. The method of any one of claims 20 to 36 wherein the mammalian animal is a human.

38. The method of any one of claims 20 to 36 wherein the mammalian animal is a livestock animal, laboratory test animal or a companion animal.

39. A substantially homogeneous population of APCs or IPAs from the CNS such as brain including retina tissue prepared by the method comprising subjecting said CNS tissue or part thereof to tissue disruptive means and contacting said immobilized tissue with interactive molecules to a cell marker selectively present or absent on or in said APCs or IPAs to generate a population comprising at least APCs and/or EPAs and then contacting the isolated cells with an interactive molecule to at least one other cell marker specific for either said APCs or IPAs or specific for a cell marker absent from either APCs or IPAs to selectively remove or retain the desired cell type.

40. A method of cell replacement therapy in a mammalian animal, said method comprising generating a substantially homogeneous population of lineage-specific cells and introducing same into an organ or tissue requiring cells to be replaced or to another location from where the cells can migrate to an organ or tissue requiring cells wherein the introduced cells are subject to expansion or proliferation in vitro and/or in vivo by one or more growth factors.

41. The method of claim 40 wherein the lineage-specific cells are astrocyte precursor cells (APCs).

42. The method of claim 40 wherein the lineage-specific cells are immature perinatal astrocytes (IPAs).

43. The method of claim 40 wherein the lineage-specific cells are mature perinatal astrocytes (MPAs).

44. The method of claim 40 wherein the lineage-specific cells are adult astrocytes.

45. The method of claim 40 wherein the mammalian animal is at a prenatal stage.

46. The method of claim 40 wherein the mammalian animal is at a postnatal stage.

47. The method of claim 46 wherein the postnatal animal is an adult.

48. The method of claim 40 wherein the separation discrimination means is based on a different range of cell markers present at different developmental stages of the lineage-specific cells.

49. The method of claim 48 wherein the separation discrimination means is based on positive or negative selection of cell surface markers.

50. The method of claim 48 or 49 wherein the neuronal cells are removed by negative selection with N-CAM.

51. The method of claim 48 or 49 or 50 wherein glial cells are selected positively using one or more of A2B5, GD3, 3CB2, FGFR3, PDGFRα or a combination thereof.

52. The method of claim 48 or 49 or 50 or 51 wherein oligodendrocytes are removed by negative selection with one or more of Gal-C, Glc, 01, 04, anti-Mog or NG2 or a combination thereof.

53. The method of claim 48 wherein the cells are sorted by recognition of cell markers by immunological reagents.

54. The method of claim 53 wherein the immunoglobulin reagents are antibodies.

55. The method of claim 40 wherein the cells isolated are immunohistochemically Pax2⁺ GFAP⁻.

56. The method of claim 40 wherein the cells isolated are immunohistochemically Pax2⁺ GFAP⁺.

57. The method of claim 40 wherein IPAs are isolated by the immunological separation of a population of vimentin⁺ cells and then isolating GFAP⁺ cells from said vimentin⁺ population.

58. The method of any one of claims 48 to 57 wherein the mammalian animal is a human.

59. The method of any one of claims 40 to 58 wherein the mammalian animal is a livestock animal, laboratory test animal or a companion animal.

60. The method of claim 40 wherein the therapy is for a degenerative disorder.

61. The method of claim 60 wherein the degenerative disorder is Alzheimer's disease, Huntington's disease, HIV-associated dementia (HIV-D), a chronic neurological disorder, Parkinson's disease, epilepsy, stroke or alcoholism.

62. The method of claim 60 wherein the degenerative disorder is hypoxia or a spinal chord injury.

63. The method of claim 40 wherein the therapy is an acute brain injury or CNS dysfunction.

64. The method of any one of claims 40 to 63 wherein the lineage-specific cells are co-introduced with neural stem cells or neuronal cells.

65. The method of claims 40 to 63 wherein the lineage specific cells are from the same subject being treated.

66. The method of any one of claims 40 to 63 wherein the lineage-specific cells are from a different subject being tested.

67. A method for assessing the level of healthy tissue in a CNS biopsy such as a brain biopsy in an adult subject said method comprising determining in said biopsy presence of Pax2⁺ astrocyte cells wherein the presence of said Pax2⁺ cells is indicative of a reversion in the maturation of said astrocytes.

68. A composition of astrocyte precursor cells such as APCs or IPAs in substantially homogeneous form, said composition optionally further comprising one or more pharmaceutically-acceptable carriers and/or diluents.

69. Conditioned medium from the in vitro culture of astrocyte precursor cells such as APCs or IPAs wherein said conditioned medium comprises one or more growth factors or autocrine factors.

70. A growth or autocrine factor obtainable from conditioned medium of an in vitro cell culture of astrocyte precursors such as APCs or IPAs.

71. A method for generating a substantially homogenous population of mammalian cells of the astrocytic lineage, said method comprising isolating cell suspension from an adult brain or embryonic brain and removing neural precursor cells by a negative selection using N-CAM or functional equivalent and then selecting positively for glial cells using one or more of A2B5, GD3, 3CB2, FGFR3, PDGFRα or a combination thereof or a functional equivalent thereof; culturing the resulting cells in a serum free medium together with a growth factor and removing by negative selection oligodendrocytes using markers Glc, Gal-C, 01, 04, anti-Mog or a combination thereof or a functional equivalent thereof and then inducing differentiation along the maturation pathway by culturing cells in the presence of one or more of CNTF, LIF, BMP such as BMP4, cAMP, TGFβ and/or EGF or functional equivalents thereof.

72. The method of claim 71 wherein the serum free medium is DMEM/F-12.

73. The method of claim 72 wherein the DMEM/F-12 medium further comprises a growth factor selected from bFGF and chick embryo extract.

74. Cells of the astrocytic lineage isolated by the method of any one of claims 71 to 73.
